SEQUENCE LISTING (45 sequences)

SEQ ID NO: 1 - protein, 214 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 2 - protein, 222 aa, Bos taurus
MKFFIFTCLLAVALAKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 3 - protein, 224 aa, Bos taurus
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 4 - protein, 178 aa, Bos taurus
MKCLLLALALTCGAQALIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 5 - protein, 186 aa, Synthetic construct
MKFFAIAALFAAAAVAQPNVISKRLIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 6 - protein, 142 aa, Bos taurus
MMSFVSLLLVGILFHATQAEQLTKCEVFRELKDLKGYGGVSLPEWVCTTFHTSGYDTQAIVQNNDSTEYGLFQINNKIWCKDDQNPHSSNICNISCDKFLDDDLTDDIMCVKKILDKVGINYWLAHKALCSEKLDQWLCEKL

SEQ ID NO: 7 - protein, 194 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSF

SEQ ID NO: 8 - protein, 189 aa, Bos taurus
MKFFIFTCLLAVALAKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKF

SEQ ID NO: 9 - protein, 220 aa, Bos taurus
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPF

SEQ ID NO: 10 - protein, 167 aa, Bos taurus
MKCLLLALALTCGAQALIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSF

SEQ ID NO: 11 - protein, 175 aa, Synthetic construct
MKFFAIAALFAAAAVAQPNVISKRLIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSF

SEQ ID NO: 12 - protein, 99 aa, Bos taurus
MMSFVSLLLVGILFHATQAEQLTKCEVFRELKDLKGYGGVSLPEWVCTTFHTSGYDTQAIVQNNDSTEYGLFQINNKIWCKDDQNPHSSNICNISCDKF

SEQ ID NO: 13 - protein, 190 aa, Bos taurus
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 14 - protein, 64 aa, Bos taurus
MAIPPKKNQDKTEIPTINTIASGEPTSTPTTEAVESTVATLEDSPEVIESPPEINTVQVTSTAV

SEQ ID NO: 15 - protein, 63 aa, Bos taurus
AIPPKKNQDKTEIPTINTIASGEPTSTPTTEAVESTVATLEDSPEVIESPPEINTVQVTSTAV

SEQ ID NO: 16 - protein, 258 aa, Synthetic construct
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFMAIPPKKNQDKTEIPTINTIASGEPTSTPTTEAVESTVATLEDSPEVIESPPEINTVQVTSTAV

SEQ ID NO: 17 - protein, 253 aa, Synthetic construct
MKFFIFTCLLAVALAKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFMAIPPKKNQDKTEIPTINTIASGEPTSTPTTEAVESTVATLEDSPEVIESPPEINTVQVTSTAV

SEQ ID NO: 18 - protein, 284 aa, Synthetic construct
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFMAIPPKKNQDKTEIPTINTIASGEPTSTPTTEAVESTVATLEDSPEVIESPPEINTVQVTSTAV

SEQ ID NO: 19 - protein, 231 aa, Synthetic construct
MKCLLLALALTCGAQALIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFMAIPPKKNQDKTEIPTINTIASGEPTSTPTTEAVESTVATLEDSPEVIESPPEINTVQVTSTAV

SEQ ID NO: 20 - protein, 239 aa, Synthetic construct
MKFFAIAALFAAAAVAQPNVISKRLIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFMAIPPKKNQDKTEIPTINTIASGEPTSTPTTEAVESTVATLEDSPEVIESPPEINTVQVTSTAV

SEQ ID NO: 21 - protein, 163 aa, Synthetic construct
MMSFVSLLLVGILFHATQAEQLTKCEVFRELKDLKGYGGVSLPEWVCTTFHTSGYDTQAIVQNNDSTEYGLFQINNKIWCKDDQNPHSSNICNISCDKFMAIPPKKNQDKTEIPTINTIASGEPTSTPTTEAVESTVATLEDSPEVIESPPEINTVQVTSTAV

SEQ ID NO: 22 - DNA, 645 bp, Bos taurus
  source 1..645: mol_type = other DNA
atgaaacttctcatccttacctgtcttgtggctgttgctcttgctaggcctaaacatcctatcaagcaccaaggactccctcaagaagtcctcaatgaaaatttactcaggttttttgtggcaccttttccagaagtgtttggaaaggagaaggtcaatgaactgagcaaggatattgggagtgaatcaactgaggatcaagccatggaagatattaagcaaatggaagctgaaagcatttcgtcaagtgaggaaattgttcccaatagtgttgagcagaagcacattcaaaaggaagatgtgccctctgagcgttacctgggttatctggaacagcttctcagactgaaaaaatacaaagtaccccagctggaaattgttcccaatagtgctgaggaacgacttcacagtatgaaagagggaatccatgcccaacagaaagaacctatgataggagtgaatcaggaactggcctacttctaccctgagcttttcagacaattctaccagctggatgcctatccatctggtgcctggtattacgttccactaggcacacaatacactgatgccccatcattctctgacatccctaatcctattggctctgagaacagtgaaaagactactatgccactgtggtga

SEQ ID NO: 23 - DNA, 669 bp, Bos taurus
  source 1..669: mol_type = other DNA
atgaagttcttcatctttacctgccttttggctgttgcccttgcaaagaatacgatggaacatgtctcctccagtgaggaatctatcatctcccaggaaacatataagcaggaaaagaatatggccattaatcccagcaaggagaacctttgctccacattctgcaaggaagttgtaaggaacgcaaatgaagaggaatattctatcggctcatctagtgaggaatctgctgaagttgccactgaggaagttaagattactgtggacgataagcactaccagaaagcactgaatgaaatcaatcagttttatcagaagttcccccagtatctccagtatctgtatcaaggtccaattgttttgaacccatgggatcaggttaagaggaatgctgttcccattactcccactctgaacagagagcagctctccaccagtgaggaaaattcaaagaagaccgttgacatggaatcaacagaagtattcactaagaaaactaaactgactgaagaagaaaagaatcgcctaaattttctgaaaaaaatcagccagcgttaccagaaattcgccttgccccagtatctcaagactgtttatcagcatcagaaagctatgaagccatggattcaacctaagacaaaggttattccctatgtgaggtacctttaa

SEQ ID NO: 24 - DNA, 675 bp, Bos taurus
  source 1..675: mol_type = other DNA
atgaaggtcctcatccttgcctgcctggtggctctggcccttgcaagagagctggaagaactcaatgtacctggtgagattgtggaaagcctttcaagcagcgaggaatcaattacacgcatcaataagaaaattgagaagtttcagagtgaggaacagcagcaaacagaggatgaactccaggataaaatccacccctttgcccagacacagtctctagtctatcccttccctgggcccatccctaacagcctcccacaaaacatccctcctcttactcaaacccctgtggtggtgccgcctttccttcagcctgaagtaatgggagtctccaaagtgaaggaggctatggcccctaagcacaaagaaatgcccttccctaaatatccagttgagccctttactgaaagccagagcctgactctcactgatgttgaaaatctgcaccttcctctgcctctgctccagtcttggatgcaccagcctcaccagcctcttcctccaactgtcatgtttcctcctcagtccgtgctgtccctttctcagtccaaagtcctgcctgttccccagaaagcagtgccctatccccagagagatatgcccattcaggcctttctgctgtaccaggagcctgtactcggtcctgtccggggacccttccctattattgtctaa

SEQ ID NO: 25 - DNA, 534 bp, Bos taurus
  source 1..534: mol_type = other DNA
atgaagtgcctcctgcttgccctggccctcacttgtggcgcccaggccctcattgtcacccagaccatgaagggcctggatatccagaaggtggcggggacttggtactccttggccatggcggccagcgacatctccctgctggacgcccagagtgcccccctgagagtgtatgtggaggagctgaagcccacccctgagggcgacctggagatcctgctgcagaaatgggagaacggtgagtgtgctcagaagaagatcattgcagaaaaaaccaagatccctgcggtgttcaagatcgatgccttgaatgagaacaaagtccttgtgctggacaccgactacaaaaagtacctgctcttctgcatggagaacagtgctgagcccgagcaaagcctggcctgccagtgcctggtcaggaccccggaggtggacgacgaggccctggagaaattcgacaaagccctcaaggccctgcccatgcacatccggctgtccttcaacccaacccagctggaggagcagtgccacatc

SEQ ID NO: 26 - DNA, 558 bp, Synthetic construct
  source 1..558: mol_type = other DNA
atgaagtttttcgccattgccgccctatttgccgccgctgccgttgcccagcctaacgtcatctcgaagcggctcatcgtcacccagaccatgaagggcctcgacatccagaaggtcgccggcacctggtacagcctcgccatggccgccagcgacatcagcctgctcgacgcccagagcgcccctctccgcgtctacgtcgaggagctgaagcccacgcctgagggcgacctcgagatcctcctgcagaagtgggagaacggcgagtgcgcccagaagaagatcattgccgagaagacgaagatccccgccgtgttcaagatcgacgccctcaacgagaacaaggtcctcgtcctcgacaccgactacaagaagtacctcctgttctgcatggagaactccgccgagcctgagcagagcctcgcctgccagtgcctcgttcgcacgcccgaggtcgacgacgaggccctcgagaagttcgacaaggccctcaaggctctccccatgcacatccgcctcagcttcaaccccacgcagctcgaggagcagtgccacatc

SEQ ID NO: 27 - DNA, 429 bp, Bos taurus
  source 1..429: mol_type = other DNA
atgatgtcctttgtctctctgctcctggtaggcatcctattccatgccacccaggctgaacagttaacaaaatgtgaggtgttccgggagctgaaagacttgaagggctacggaggtgtcagtttgcctgaatgggtctgtaccacgtttcataccagtggttatgacacacaagccatagtacaaaacaatgacagcacagaatatggactcttccagataaataataaaatttggtgcaaagacgaccagaaccctcactcaagcaacatctgtaacatctcctgtgacaagttcctggatgatgatcttactgatgacattatgtgtgtcaagaagattctggataaagtaggaattaactactggttggcccataaagcactctgttctgagaagctggatcagtggctctgtgagaagttgtga

SEQ ID NO: 28 - DNA, 582 bp, Bos taurus
  source 1..582: mol_type = other DNA
atgaaacttctcatccttacctgtcttgtggctgttgctcttgctaggcctaaacatcctatcaagcaccaaggactccctcaagaagtcctcaatgaaaatttactcaggttttttgtggcaccttttccagaagtgtttggaaaggagaaggtcaatgaactgagcaaggatattgggagtgaatcaactgaggatcaagccatggaagatattaagcaaatggaagctgaaagcatttcgtcaagtgaggaaattgttcccaatagtgttgagcagaagcacattcaaaaggaagatgtgccctctgagcgttacctgggttatctggaacagcttctcagactgaaaaaatacaaagtaccccagctggaaattgttcccaatagtgctgaggaacgacttcacagtatgaaagagggaatccatgcccaacagaaagaacctatgataggagtgaatcaggaactggcctacttctaccctgagcttttcagacaattctaccagctggatgcctatccatctggtgcctggtattacgttccactaggcacacaatacactgatgccccatcattc

SEQ ID NO: 29 - DNA, 567 bp, Bos taurus
  source 1..567: mol_type = other DNA
atgaagttcttcatctttacctgccttttggctgttgcccttgcaaagaatacgatggaacatgtctcctccagtgaggaatctatcatctcccaggaaacatataagcaggaaaagaatatggccattaatcccagcaaggagaacctttgctccacattctgcaaggaagttgtaaggaacgcaaatgaagaggaatattctatcggctcatctagtgaggaatctgctgaagttgccactgaggaagttaagattactgtggacgataagcactaccagaaagcactgaatgaaatcaatcagttttatcagaagttcccccagtatctccagtatctgtatcaaggtccaattgttttgaacccatgggatcaggttaagaggaatgctgttcccattactcccactctgaacagagagcagctctccaccagtgaggaaaattcaaagaagaccgttgacatggaatcaacagaagtattcactaagaaaactaaactgactgaagaagaaaagaatcgcctaaattttctgaaaaaaatcagccagcgttaccagaaattc

SEQ ID NO: 30 - DNA, 660 bp, Bos taurus
  source 1..660: mol_type = other DNA
atgaaggtcctcatccttgcctgcctggtggctctggcccttgcaagagagctggaagaactcaatgtacctggtgagattgtggaaagcctttcaagcagcgaggaatcaattacacgcatcaataagaaaattgagaagtttcagagtgaggaacagcagcaaacagaggatgaactccaggataaaatccacccctttgcccagacacagtctctagtctatcccttccctgggcccatccctaacagcctcccacaaaacatccctcctcttactcaaacccctgtggtggtgccgcctttccttcagcctgaagtaatgggagtctccaaagtgaaggaggctatggcccctaagcacaaagaaatgcccttccctaaatatccagttgagccctttactgaaagccagagcctgactctcactgatgttgaaaatctgcaccttcctctgcctctgctccagtcttggatgcaccagcctcaccagcctcttcctccaactgtcatgtttcctcctcagtccgtgctgtccctttctcagtccaaagtcctgcctgttccccagaaagcagtgccctatccccagagagatatgcccattcaggcctttctgctgtaccaggagcctgtactcggtcctgtccggggacccttc

SEQ ID NO: 31 - DNA, 501 bp, Bos taurus
  source 1..501: mol_type = other DNA
atgaagtgcctcctgcttgccctggccctcacttgtggcgcccaggccctcattgtcacccagaccatgaagggcctggatatccagaaggtggcggggacttggtactccttggccatggcggccagcgacatctccctgctggacgcccagagtgcccccctgagagtgtatgtggaggagctgaagcccacccctgagggcgacctggagatcctgctgcagaaatgggagaacggtgagtgtgctcagaagaagatcattgcagaaaaaaccaagatccctgcggtgttcaagatcgatgccttgaatgagaacaaagtccttgtgctggacaccgactacaaaaagtacctgctcttctgcatggagaacagtgctgagcccgagcaaagcctggcctgccagtgcctggtcaggaccccggaggtggacgacgaggccctggagaaattcgacaaagccctcaaggccctgcccatgcacatccggctgtccttc

SEQ ID NO: 32 - DNA, 525 bp, Synthetic construct
  source 1..525: mol_type = other DNA
atgaagtttttcgccattgccgccctatttgccgccgctgccgttgcccagcctaacgtcatctcgaagcggctcatcgtcacccagaccatgaagggcctcgacatccagaaggtcgccggcacctggtacagcctcgccatggccgccagcgacatcagcctgctcgacgcccagagcgcccctctccgcgtctacgtcgaggagctgaagcccacgcctgagggcgacctcgagatcctcctgcagaagtgggagaacggcgagtgcgcccagaagaagatcattgccgagaagacgaagatccccgccgtgttcaagatcgacgccctcaacgagaacaaggtcctcgtcctcgacaccgactacaagaagtacctcctgttctgcatggagaactccgccgagcctgagcagagcctcgcctgccagtgcctcgttcgcacgcccgaggtcgacgacgaggccctcgagaagttcgacaaggccctcaaggctctccccatgcacatccgcctcagcttc

SEQ ID NO: 33 - DNA, 297 bp, Bos taurus
  source 1..297: mol_type = other DNA
atgatgtcctttgtctctctgctcctggtaggcatcctattccatgccacccaggctgaacagttaacaaaatgtgaggtgttccgggagctgaaagacttgaagggctacggaggtgtcagtttgcctgaatgggtctgtaccacgtttcataccagtggttatgacacacaagccatagtacaaaacaatgacagcacagaatatggactcttccagataaataataaaatttggtgcaaagacgaccagaaccctcactcaagcaacatctgtaacatctcctgtgacaagttc

SEQ ID NO: 34 - DNA, 573 bp, Bos taurus
  source 1..573: mol_type = other DNA
atgatgaagagttttttcctagttgtgactatcctggcattaaccctgccatttttgggtgcccaggagcaaaaccaagaacaaccaatacgctgtgagaaagatgaaagattcttcagtgacaaaatagccaaatatatcccaattcagtatgtgctgagtaggtatcctagttatggactcaattactaccaacagaaaccagttgcactaattaataatcaatttctgccatacccatattatgcaaagccagctgcagttaggtcacctgcccaaattcttcaatggcaagttttgtcaaatactgtgcctgccaagtcctgccaagcccagccaactaccatggcacgtcacccacacccacatttatcatttatggccattccaccaaagaaaaatcaggataaaacagaaatccctaccatcaataccattgctagtggtgagcctacaagtacacctaccaccgaagcagtagagagcactgtagctactctagaagattctccagaagttattgagagcccacctgagatcaacacagtccaagttacttcaactgcagtctaa

SEQ ID NO: 35 - DNA, 192 bp, Bos taurus
  source 1..192: mol_type = other DNA
atggccattccaccaaagaaaaatcaggataaaacagaaatccctaccatcaataccattgctagtggtgagcctacaagtacacctaccaccgaagcagtagagagcactgtagctactctagaagattctccagaagttattgagagcccacctgagatcaacacagtccaagttacttcaactgcagtc

SEQ ID NO: 36 - DNA, 192 bp, Bos taurus; identical to SEQ ID NO: 35
  source 1..192: mol_type = other DNA

SEQ ID NO: 37 - DNA, 774 bp, Synthetic construct
  source 1..774: mol_type = other DNA
atgaaacttctcatccttacctgtcttgtggctgttgctcttgctaggcctaaacatcctatcaagcaccaaggactccctcaagaagtcctcaatgaaaatttactcaggttttttgtggcaccttttccagaagtgtttggaaaggagaaggtcaatgaactgagcaaggatattgggagtgaatcaactgaggatcaagccatggaagatattaagcaaatggaagctgaaagcatttcgtcaagtgaggaaattgttcccaatagtgttgagcagaagcacattcaaaaggaagatgtgccctctgagcgttacctgggttatctggaacagcttctcagactgaaaaaatacaaagtaccccagctggaaattgttcccaatagtgctgaggaacgacttcacagtatgaaagagggaatccatgcccaacagaaagaacctatgataggagtgaatcaggaactggcctacttctaccctgagcttttcagacaattctaccagctggatgcctatccatctggtgcctggtattacgttccactaggcacacaatacactgatgccccatcattcatggccattccaccaaagaaaaatcaggataaaacagaaatccctaccatcaataccattgctagtggtgagcctacaagtacacctaccaccgaagcagtagagagcactgtagctactctagaagattctccagaagttattgagagcccacctgagatcaacacagtccaagttacttcaactgcagtc

SEQ ID NO: 38 - DNA, 759 bp, Synthetic construct
  source 1..759: mol_type = other DNA
atgaagttcttcatctttacctgccttttggctgttgcccttgcaaagaatacgatggaacatgtctcctccagtgaggaatctatcatctcccaggaaacatataagcaggaaaagaatatggccattaatcccagcaaggagaacctttgctccacattctgcaaggaagttgtaaggaacgcaaatgaagaggaatattctatcggctcatctagtgaggaatctgctgaagttgccactgaggaagttaagattactgtggacgataagcactaccagaaagcactgaatgaaatcaatcagttttatcagaagttcccccagtatctccagtatctgtatcaaggtccaattgttttgaacccatgggatcaggttaagaggaatgctgttcccattactcccactctgaacagagagcagctctccaccagtgaggaaaattcaaagaagaccgttgacatggaatcaacagaagtattcactaagaaaactaaactgactgaagaagaaaagaatcgcctaaattttctgaaaaaaatcagccagcgttaccagaaattcatggccattccaccaaagaaaaatcaggataaaacagaaatccctaccatcaataccattgctagtggtgagcctacaagtacacctaccaccgaagcagtagagagcactgtagctactctagaagattctccagaagttattgagagcccacctgagatcaacacagtccaagttacttcaactgcagtc

SEQ ID NO: 39 - DNA, 852 bp, Synthetic construct
  source 1..852: mol_type = other DNA
atgaaggtcctcatccttgcctgcctggtggctctggcccttgcaagagagctggaagaactcaatgtacctggtgagattgtggaaagcctttcaagcagcgaggaatcaattacacgcatcaataagaaaattgagaagtttcagagtgaggaacagcagcaaacagaggatgaactccaggataaaatccacccctttgcccagacacagtctctagtctatcccttccctgggcccatccctaacagcctcccacaaaacatccctcctcttactcaaacccctgtggtggtgccgcctttccttcagcctgaagtaatgggagtctccaaagtgaaggaggctatggcccctaagcacaaagaaatgcccttccctaaatatccagttgagccctttactgaaagccagagcctgactctcactgatgttgaaaatctgcaccttcctctgcctctgctccagtcttggatgcaccagcctcaccagcctcttcctccaactgtcatgtttcctcctcagtccgtgctgtccctttctcagtccaaagtcctgcctgttccccagaaagcagtgccctatccccagagagatatgcccattcaggcctttctgctgtaccaggagcctgtactcggtcctgtccggggacccttcatggccattccaccaaagaaaaatcaggataaaacagaaatccctaccatcaataccattgctagtggtgagcctacaagtacacctaccaccgaagcagtagagagcactgtagctactctagaagattctccagaagttattgagagcccacctgagatcaacacagtccaagttacttcaactgcagtc

SEQ ID NO: 40 - DNA, 693 bp, Synthetic construct
  source 1..693: mol_type = other DNA
atgaagtgcctcctgcttgccctggccctcacttgtggcgcccaggccctcattgtcacccagaccatgaagggcctggatatccagaaggtggcggggacttggtactccttggccatggcggccagcgacatctccctgctggacgcccagagtgcccccctgagagtgtatgtggaggagctgaagcccacccctgagggcgacctggagatcctgctgcagaaatgggagaacggtgagtgtgctcagaagaagatcattgcagaaaaaaccaagatccctgcggtgttcaagatcgatgccttgaatgagaacaaagtccttgtgctggacaccgactacaaaaagtacctgctcttctgcatggagaacagtgctgagcccgagcaaagcctggcctgccagtgcctggtcaggaccccggaggtggacgacgaggccctggagaaattcgacaaagccctcaaggccctgcccatgcacatccggctgtccttcatggccattccaccaaagaaaaatcaggataaaacagaaatccctaccatcaataccattgctagtggtgagcctacaagtacacctaccaccgaagcagtagagagcactgtagctactctagaagattctccagaagttattgagagcccacctgagatcaacacagtccaagttacttcaactgcagtc

SEQ ID NO: 41 - DNA, 717 bp, Synthetic construct
  source 1..717: mol_type = other DNA
atgaagtttttcgccattgccgccctatttgccgccgctgccgttgcccagcctaacgtcatctcgaagcggctcatcgtcacccagaccatgaagggcctcgacatccagaaggtcgccggcacctggtacagcctcgccatggccgccagcgacatcagcctgctcgacgcccagagcgcccctctccgcgtctacgtcgaggagctgaagcccacgcctgagggcgacctcgagatcctcctgcagaagtgggagaacggcgagtgcgcccagaagaagatcattgccgagaagacgaagatccccgccgtgttcaagatcgacgccctcaacgagaacaaggtcctcgtcctcgacaccgactacaagaagtacctcctgttctgcatggagaactccgccgagcctgagcagagcctcgcctgccagtgcctcgttcgcacgcccgaggtcgacgacgaggccctcgagaagttcgacaaggccctcaaggctctccccatgcacatccgcctcagcttcatggccatcccccccaagaagaaccaggacaagaccgagatccccaccatcaacaccatcgcctccggcgagcccacctccacccccaccaccgaggccgtcgagtccaccgtcgccaccctggaggactcccccgaggtcatcgagtccccccccgagatcaacaccgtccaggtcacctccaccgccgtc

SEQ ID NO: 42 - DNA, 489 bp, Synthetic construct
  source 1..489: mol_type = other DNA
atgatgtcctttgtctctctgctcctggtaggcatcctattccatgccacccaggctgaacagttaacaaaatgtgaggtgttccgggagctgaaagacttgaagggctacggaggtgtcagtttgcctgaatgggtctgtaccacgtttcataccagtggttatgacacacaagccatagtacaaaacaatgacagcacagaatatggactcttccagataaataataaaatttggtgcaaagacgaccagaaccctcactcaagcaacatctgtaacatctcctgtgacaagttcatggccattccaccaaagaaaaatcaggataaaacagaaatccctaccatcaataccattgctagtggtgagcctacaagtacacctaccaccgaagcagtagagagcactgtagctactctagaagattctccagaagttattgagagcccacctgagatcaacacagtccaagttacttcaactgcagtc

SEQ ID NO: 43 - DNA, 486 bp, Bos taurus
  source 1..486: mol_type = other DNA
ctcatcgtcacccagaccatgaagggcctcgacatccagaaggtcgccggcacctggtacagcctcgccatggccgccagcgacatcagcctgctcgacgcccagagcgcccctctccgcgtctacgtcgaggagctgaagcccacgcctgagggcgacctcgagatcctcctgcagaagtgggagaacggcgagtgcgcccagaagaagatcattgccgagaagacgaagatccccgccgtgttcaagatcgacgccctcaacgagaacaaggtcctcgtcctcgacaccgactacaagaagtacctcctgttctgcatggagaactccgccgagcctgagcagagcctcgcctgccagtgcctcgttcgcacgcccgaggtcgacgacgaggccctcgagaagttcgacaaggccctcaaggctctccccatgcacatccgcctcagcttcaaccccacgcagctcgaggagcagtgccacatc

SEQ ID NO: 44 - DNA, 453 bp, Bos taurus
  source 1..453: mol_type = other DNA
ctcatcgtcacccagaccatgaagggcctcgacatccagaaggtcgccggcacctggtacagcctcgccatggccgccagcgacatcagcctgctcgacgcccagagcgcccctctccgcgtctacgtcgaggagctgaagcccacgcctgagggcgacctcgagatcctcctgcagaagtgggagaacggcgagtgcgcccagaagaagatcattgccgagaagacgaagatccccgccgtgttcaagatcgacgccctcaacgagaacaaggtcctcgtcctcgacaccgactacaagaagtacctcctgttctgcatggagaactccgccgagcctgagcagagcctcgcctgccagtgcctcgttcgcacgcccgaggtcgacgacgaggccctcgagaagttcgacaaggccctcaaggctctccccatgcacatccgcctcagcttc

SEQ ID NO: 45 - DNA, 72 bp, Synthetic construct
  source 1..72: mol_type = other DNA
atgaagtttttcgccattgccgccctatttgccgccgctgccgttgcccagcctaacgtcatctcgaagcgg

(19) United States
(12) Patent Application Publication
(10) Pub. No.: US 2026/0022148 A1
(43) Pub. Date: Jan. 22, 2026

(54) CHIMERIC CASEIN AND WHEY PROTEINS AND USE THEREOF

(71) Applicant: IMAGINDAIRY LTD., Ashdod (IL)

(72) Inventors: Arie ABO, Emeryville, CA (US); Ariella LAVY-ALPEROVITCH, Haifa (IL)

(21) Appl. No.: 19/101,683

(22) PCT Filed: Aug. 15, 2023
(86) PCT No.: PCT/IL2023/050858

§ 371 Date: Feb. 6, 2025

Related U.S. Application Data
(60) Provisional application No. 63/398,022, filed on Aug. 15, 2022.

(51) Int. Cl. C07K 14/47; A23C 11/02; C12N 9/64; C12N 15/81

(52) U.S. Cl. CPC C07K 14/4717; A23C 11/02; C12N 9/6483; C12N 15/81; C07K 2319/50; C12Y 304/23004

(57) ABSTRACT

The present invention provides a chimeric polynucleotide comprising: a first nucleic acid sequence encoding a first polypeptide selected from: alpha S1 casein, alpha S2 casein, and beta casein, a whey polypeptide, or a functional analog thereof, a second nucleic acid sequence encoding a cleavage site of a proteolytic enzyme; and, a third nucleic acid sequence encoding a kappa casein macropeptide, Further provided are a chimeric polypeptide, as well as compositions comprising same, and methods of using same, such as for producing an edible composition comprising casein and/or whey polypeptide aggregates, such as a dairy substituent curd.

### REFERENCE TO AN ELECTRONIC SEQUENCE LISTING

The contents of the electronic sequence listing (IMD-P-004-PCT ST26.xml; size: 56,277 bytes; and date of creation: Jul. 18, 2023) is herein incorporated by reference in its entirety.

### CROSS-REFERENCE TO RELATED APPLICATIONS

This application claims the benefit of priority of U.S. Provisional Application No. 63/398,022, titled “CHIMERIC CASEIN AND WHEY PROTEINS AND USE THEREOF”, filed 15 Aug. 2022, the content of which are incorporated herein by reference in their entirety.

### FIELD OF INVENTION

The present invention is in the field of producing dairy-like compositions.

### BACKGROUND OF THE INVENTION

Casein and whey protein are the major proteins of milk. Casein constitutes approximately 80% of the total protein in bovine milk, whereas whey protein accounts for about 20%. The major whey protein is β-lactoglobulin (BLG), followed by α-lactalbumin (ALA). β-lactoglobulin constitutes about 65% from whey protein, and α-lactalbumin around 25%.

K Casein proteins are the dominant protein group in bovine milk and are the major functional contributor for curd formation that is essential for dairy products. The caseins are nature-designed to be dispersed in an aqueous solvent, carry relatively large quantities of calcium and calcium phosphate, and still maintain a low viscosity at ˜2.5% (w/w). Casein proteins are phosphorylated, and glycosylated complex synthesized by mammary glands. It is constituted of 4 different polypeptide chains (αs1, αs2, and β and κ casein) held together by noncovalent interactions. Casein fractions are organized in micellar aggregates that also contain bivalent cations (calcium and smaller amounts of magnesium), ranging 20-300 nm in diameter. This structure allows highly stable dispersion of hydrophobic fractions in a colloidal state by the action of hydrophilic bonds. Bovine K-caseins can be cleaved by chymosin (or rennet), and converted to an insoluble peptide, named para-K-casein, accompanied by the release of a soluble peptide containing carbohydrates termed glycomacropeptide (or κ casein macropeptide).

By reduction the pH to 4.6 together with a treatment of rennet (protease chymosin) the casein micelles aggregate to form a unique matrix and texture that is essential for dairy products such as cheese and yogurt. This unique matrix could be achieved by the combination of K casein with only one of the other casein αs1, αs2, B, or with one of the whey proteins.

### SUMMARY OF THE INVENTION

According to one aspect there is provided a chimeric polynucleotide comprising: a first nucleic acid sequence encoding a first polypeptide selected from the group consisting of: alpha S1 casein, alpha S2 casein, beta casein, whey polypeptide, and any functional analog thereof; a second nucleic acid sequence encoding a cleavage site of a proteolytic enzyme; and a third nucleic acid sequence encoding a kappa casein macropeptide, wherein the second nucleic acid sequence is positioned between the first and third nucleic acid sequences, and the first, second, and third nucleic acid sequences are operably linked.

In some embodiments, the whey polypeptide comprises beta lactoglobulin or alpha lactalbumin.

In some embodiments, the proteolytic enzyme belongs to the family of aspartic proteases.

In some embodiments, the proteolytic enzyme comprises a chymosin.

In some embodiments, the casein comprises a bovine casein.

In some embodiments, the whey polypeptide comprises a bovine or a fungal whey polypeptide.

In some embodiments, the chimeric polynucleotide comprises a codon optimized sequence for expression in a cell.

In some embodiments, the cell is a transfected or a transgenic cell.

In some embodiments, the chimeric polynucleotide comprises the nucleotide sequence set forth in any one of SEQ ID Nos: 37-42.

According to another aspect, there is provided a plasmid comprising the chimeric polynucleotide.

In some embodiments, the plasmid is an expression vector.

In some embodiments the chimeric polypeptide is encoded, expressed, or both from: the chimeric polynucleotide disclosed herein; or the plasmid disclosed herein.

In some embodiments, the chimeric polypeptide comprises the amino acid sequence set forth in any one of SEQ ID Nos: 16-21.

According to another aspect, there is provided a cell, comprising any one of: (a) the chimeric polynucleotide disclosed herein; (b) the plasmid disclosed herein; (c) the chimeric polypeptide disclosed herein; and any combination of (a) to (c).

According to another aspect there is provided a composition comprising any one of: (a) the chimeric polynucleotide disclosed herein; (b) the plasmid disclosed herein; (c) the chimeric polypeptide disclosed herein; (d) the cell disclosed herein; and any combination of (a) to (d); and an acceptable carrier.

In some embodiments, the composition further comprises any one of: calcium salt, magnesium salt, and both.

In some embodiments, the composition disclosed herein is an edible composition.

In some embodiments, the edible composition is a dairy product substituent.

According to another aspect there is provided a method for producing an edible composition comprising casein and/or whey polypeptide aggregates, the method comprising providing a composition comprising a chimeric polypeptide comprising: (i) a first polypeptide being a polypeptide selected from the group consisting of: alpha S1 casein, alpha S2 casein, beta casein, whey polypeptide, whey polypeptide, and a functional analog thereof; and (ii) a second polypeptide being a kappa casein macropeptide, wherein the first polypeptide and the second polypeptide are interspaced by a cleavage site of a proteolytic enzyme, and contacting the composition with an effective amount of the proteolytic enzyme, thereby producing the edible composition comprising casein and/or whey polypeptide aggregates.

In some embodiments, the method further comprises a step proceeding said contacting, comprising isolating said casein and/or whey polypeptide aggregates.

In some embodiments, contacting is performed at pH of 4.0-6.0.

In some embodiments, the produced edible composition comprising the aggregates of casein and/or whey polypeptide, comprises a cheese or a curd.

According to another aspect there is provided an edible composition produced according to the method disclosed herein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 includes a photograph of a Coomasie stained gel separation of of different proteins produced herein on sodium dodecyl sulfate polyacrylamide gel electrophoresis (SDS-PAGE). 1, β-lactoglobulin (BLG) unfiltered; 2, BLG filtered; 3, BLG: Kappa-Casein-Glycomacropeptide unfiltered (V85); 4, V85 filtered; 5, V85 after overnight in −80° C. unfiltered; 6, sample 5 filtered; and 7-10, BLG standards (250 ng, 500 ng, 1,000 ng, and 2,000 ng, respectively).

### DETAILED DESCRIPTION OF THE INVENTION

The present invention, in some embodiments, provides a chimeric polynucleotide comprising a plurality of nucleic acids sequences encoding a plurality of: chimeric or fused casein polypeptides, or casein and whey polypeptides. In some embodiments, the chimeric polynucleotide comprises: a first nucleic acid sequence encoding a first polypeptide selected from: alpha S1 casein, alpha S2 casein, beta casein, whey polypeptide, or any functional analog thereof, a second nucleic acid sequence encoding a cleavage site of a proteolytic enzyme; and a third nucleic acid sequence encoding a kappa casein macropeptide.

Further provided are cells and vectors comprising any one of the polynucleotides, the chimeric polypeptides, as well as methods of using same, such as, but not limited to, producing a dairy product substituent.

### Chimeric Polynucleotides

According to some embodiments, there is provided a chimeric polynucleotide comprising: (a) a first nucleic acid sequence encoding a first polypeptide selected from: alpha S1 casein, alpha S2 casein, beta casein, whey polypeptide, or a functional analog thereof, (b) a second nucleic acid sequence encoding a cleavage site of a proteolytic enzyme; and (c) a third nucleic acid sequence encoding a kappa casein macropeptide.

In some embodiments, the second nucleic acid sequence is positioned between the first and third nucleic acid sequences.

In some embodiments, the first, second, and third nucleic acid sequences are operably linked.

In some embodiments, the alpha S1 casein, alpha S2 casein, beta casein, kappa casein, or a whey polypeptide arc mammalian milk proteins. In some embodiments, the chimeric polynucleotide of the invention encodes a chimeric polypeptide that forms casein and/or casein-like micelles, within an aqueous solution.

As used herein, the term “casein and/or casein-like micelles” refers to soluble structure of caseins within an aqueous solution, such as, but not limited to a dairy aqueous solution.

In some embodiments, the term “casein-like micelles” refers to a soluble structure composed of a kappa casein macropeptide and a whey polypeptide, forming a soluble structure that resembles casein micelles. In some embodiments, a whey polypeptide comprises alpha lactalbumin. In some embodiments, a whey polypeptide comprises beta lactoglobulin.

In some embodiments, casein and/or casein-like micelles comprise a structure of spherical colloidal particles. In some embodiments, the spherical colloidal particles are characterized by a diameter, maximal diameter, maximal size, average size, average diameter, ranging from 50-600 nm.

As used herein, the term “kappa casein macropeptide”, refers to the C terminus polypeptide that is formed as a result of full-length kappa casein proteolytic cleavage, such as by chymosin.

In some embodiments, a kappa casein macropeptide comprises the amino acid sequence set forth in SEQ ID NO: 14. In some embodiments, the third nucleic acid within the chimeric polynucleotide encodes a kappa casein macropeptide analog. As used herein, the term “kappa casein macropeptide analog” comprises kappa casein macropeptide, devoid of the first amino acid Met. In some embodiments, a kappa casein macropeptide analog comprises the amino acid sequence set forth in SEQ ID NO: 15.

In some embodiments, the chimeric nucleic acid comprises a first nucleic acid sequence encoding alpha S1 casein, a second nucleic acid sequence encoding a cleavage site of a proteolytic enzyme, and a third nucleic acid encoding a kappa casein macropeptide. In some embodiments, the chimeric nucleotide comprises a first nucleic acid encoding alpha S2 casein, a second nucleic acid encoding a cleavage site of a proteolytic enzyme, and a third nucleic acid encoding a kappa casein macropeptide. In some embodiments, the chimeric polypeptide comprises a first nucleic acid encoding a beta casein, a second nucleic acid encoding a cleavage site of a proteolytic enzyme, and a third nucleic acid encoding a kappa casein macropeptide. In some embodiments, the chimeric polypeptide comprises a first nucleic acid encoding an alpha lactalbumin, a second nucleic acid encoding a cleavage site of a proteolytic enzyme, and a third nucleic acid encoding a kappa casein macropeptide. In some embodiments, the chimeric polypeptide comprises a first nucleic acid encoding a beta lactoglobulin, a second nucleic acid encoding a cleavage site of a proteolytic enzyme, and a third nucleic acid encoding a kappa casein macropeptide. In some embodiments, the first nucleic acid encodes a first polypeptide or a functional analog thereof, of any one of: alpha S1 casein, alpha S2 casein, beta casein, beta lactoglobulin and alpha lactalbumin. As used herein, the term “a first polypeptide functional analog” refers to a polynucleotide encoding a truncated polypeptide, compared to the wild type or native polypeptide, being devoid of the amino acids located C terminally to the last Phe (F), at the C terminal end of the wildtype from of the first polypeptide.

In some embodiments, the chimeric polypeptide comprises: a first nucleic acid encoding a first polypeptide or a functional analog thereof, of any one of: alpha S1 casein, alpha S2 casein, beta casein, beta lactoglobulin and alpha lactalbumin, and a second nucleic acid encoding a kappa casein macropeptide. In some embodiments, the cleavage site within the chimeric polypeptide is located between the last amino acid of the first polypeptide, such as, but not limited to, the C′ terminal end, being Phe; and the first amino acid at the N terminal end, of the kappa casein macropeptide. In some embodiments, the first amino acid at the N terminal end is a methionine. In some embodiments, the first amino acid at the N terminal end is a methionine of SEQ ID NO: 14. In some embodiments, the first amino acid at the N terminal end is the first amino acid at the N terminal end of SEQ ID NO: 14.

In some embodiments, the first nucleic acid sequence encodes a casein selected from: alpha S1, alpha S2, beta casein or whey polypeptide. In some embodiments, any one of: alpha S1, alpha S2, beta casein, and whey polypeptide, is devoid of a signal peptide.

As used herein, the term “cleavage site” refers to a site within a polypeptide, that is cleaved, or broken down by an enzymatic proteolytic activity.

In some embodiments, the proteolytic enzyme comprises endopeptidase. In some embodiments, the polypeptide bonds are cleaved by hydrolysis. In some embodiments, the proteolytic enzyme comprises an aspartic protease (e.g., utilizes an aspartate carboxylic acid). In some embodiments the proteolytic enzyme comprises a serine protease (e.g., utilizes a serine alcohol). In some embodiments, the proteolytic enzyme comprises a cysteine protease (e.g., utilizes a cysteine thiol). In some embodiments the proteolytic enzyme comprises a threonine protease (e.g., utilizes a threonine secondary alcohol). In some embodiments, the proteolytic enzyme comprises a glutamic protease (utilizes a glutamate carboxylic acid). In some embodiments, the proteolytic enzyme comprises a metalloprotease (e.g., utilizes a metal, such as, but not limited to, zinc). In some embodiments, the proteolytic enzyme comprises an asparagine peptide lyase (e.g., utilizes an asparagine to perform an elimination reaction).

In some embodiments, the proteolytic enzyme comprises a chymosin. In some embodiments, the terms “chymosin” and “rennin” are used herein interchangeably. In some embodiments the proteolytic enzyme comprises an aspartic endopeptidase. In some embodiments, the aspartic endopeptidase belongs to MEROPS Al family. In some embodiments, the proteolytic enzyme is pepsin. In some embodiments, the proteolytic enzyme is lipase. In some embodiments, the proteolytic enzyme comprises a plurality of enzymes termed “rennet” (e.g., such as being produced in the stomachs of ruminant mammals).

In some embodiments the proteolytic enzyme is a natural enzyme present in stomachs of ruminant mammals. In some embodiments, the proteolytic enzyme is of a plant source, such as, but not limited to: ficus, galium, capparis spinosa, urtica, thistle, malva, withania coagulans, glechoma hederacea, and cynara (artichokes and cardoons). In some embodiments, the proteolytic enzyme is of a microbial source, such as but not limited to: rhizomucor michei molds. In some embodiments, the proteolytic enzyme is a synthetic enzyme produced in bacteria, fungi, or yeasts. In some embodiments, the proteolytic enzyme is a fermentation-produced chymosin (FPC), such as a chymosin produced by fungus aspergillus niger or kluyveromyces lactis.

A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

As used herein, the term “encoding” refers to molecule comprising a DNA sequence which can be transcribed into an RNA sequence which can be translated into the encoded protein or a molecule comprising the RNA sequence which can be translated into the encoded protein. In some embodiments, the molecule is a DNA molecule. In some embodiments, the molecule is an RNA molecule. In some embodiments, the DNA is cDNA. In some embodiments, the molecule is a DNA/RNA hybrid. In some embodiments, the molecule comprises non-naturally occurring nucleotides.

In some embodiments, at least one of: alpha S1 casein, alpha S2 casein, beta casein kapa casein macropeptide, beta lactoglobulin and alpha lactalbumin, are selected from: bovine, ovine, caprine, bubaline, camel, porcine, fungal proteins, or any combination thereof. In some embodiments, at least one of: alpha S1 casein, alpha S2 casein, beta casein kapa casein macropeptide, beta lactoglobulin and alpha lactalbumin, are bovine caseins. In some embodiments, whey polypeptide is a fungal polypeptide. In some embodiments, beta lactoglobulin is a fungal polypeptide. In some embodiments, a fungal polypeptide is of aspergillus oryzae.

In some embodiments, a fungal polypeptide as disclosed herein, comprises a polypeptide being heterologously synthesized by a fungus, e.g., a recombinant protein. In some embodiments, a fungal polypeptide comprises a signal peptide from a fungus.

In some embodiments, the chimeric polynucleotide comprises an optimized codon sequence for expression in a cell.

As used herein, the term “an optimized codon sequence” describes a sequence that encodes identical amino acids to those encoded by a non-optimized codon sequence (synonymous codon), however, at least one of: translation rate of the codon optimized sequence, protein product amount, duration of protein structure stability, or any combination thereof, is increased, compared to the non-optimized codon. An ordinary skill in the art will know how to optimize a codon sequence for its expression in the desired cell, using a codon optimization gene engineering tool, comprising, but not limited to, algorithms that analyze codon optimization based on the codon frequencies in the desired cell/specie. In some embodiments, increased one of: translation rate, protein product amount, and duration of structure stability, is by at least by 30%.

In some embodiments, increased one of: translation rate, protein product amount, and duration of structure stability, comprises induction by one of: 1.3-1.5 b fold, 1.6-2.0 fold, 2.1-3.0 fold, 3.1-5.0 fold, and 5.1-10.0 fold. Each possibility represents a separate embodiment of the invention.

In some embodiments, the first nucleic acid sequence encodes alpha S1 bovine casein. In some embodiments, the first nucleic acid sequence comprises the sequence:

(SEQ ID NO: 22)
ATGAAACTTCTCATCCTTACCTGTCTTGTGGCTGTTGCTCTTGCTAGGCCTAAACATCC
TATCAAGCACCAAGGACTCCCTCAAGAAGTCCTCAATGAAAATTTACTCAGGTTTTTT
GTGGCACCTTTTCCAGAAGTGTTTGGAAAGGAGAAGGTCAATGAACTGAGCAAGGAT
ATTGGGAGTGAATCAACTGAGGATCAAGCCATGGAAGATATTAAGCAAATGGAAGCT
GAAAGCATTTCGTCAAGTGAGGAAATTGTTCCCAATAGTGTTGAGCAGAAGCACATTC
AAAAGGAAGATGTGCCCTCTGAGCGTTACCTGGGTTATCTGGAACAGCTTCTCAGACT
GAAAAAATACAAAGTACCCCAGCTGGAAATTGTTCCCAATAGTGCTGAGGAACGACTT
CACAGTATGAAAGAGGGAATCCATGCCCAACAGAAAGAACCTATGATAGGAGTGAAT
CAGGAACTGGCCTACTTCTACCCTGAGCTTTTCAGACAATTCTACCAGCTGGATGCCTA
TCCATCTGGTGCCTGGTATTACGTTCCACTAGGCACACAATACACTGATGCCCCATCAT
TCTCTGACATCCCTAATCCTATTGGCTCTGAGAACAGTGAAAAGACTACTATGCCACT
GTGGTGA.

In some embodiments, the first nucleic acid sequence encodes bovine alpha S1 casein analog.

In some embodiments, nucleic acid sequence comprises the sequence:

(SEQ ID NO: 28)
ATGAAACTTCTCATCCTTACCTGTCTTGTGGCTGTTGCTCTTGCTAGGCCTAAACATCC
TATCAAGCACCAAGGACTCCCTCAAGAAGTCCTCAATGAAAATTTACTCAGGTTTTTT
GTGGCACCTTTTCCAGAAGTGTTTGGAAAGGAGAAGGTCAATGAACTGAGCAAGGAT
ATTGGGAGTGAATCAACTGAGGATCAAGCCATGGAAGATATTAAGCAAATGGAAGCT
GAAAGCATTTCGTCAAGTGAGGAAATTGTTCCCAATAGTGTTGAGCAGAAGCACATTC
AAAAGGAAGATGTGCCCTCTGAGCGTTACCTGGGTTATCTGGAACAGCTTCTCAGACT
GAAAAAATACAAAGTACCCCAGCTGGAAATTGTTCCCAATAGTGCTGAGGAACGACTT
CACAGTATGAAAGAGGGAATCCATGCCCAACAGAAAGAACCTATGATAGGAGTGAAT
CAGGAACTGGCCTACTTCTACCCTGAGCTTTTCAGACAATTCTACCAGCTGGATGCCTA
TCCATCTGGTGCCTGGTATTACGTTCCACTAGGCACACAATACACTGATGCCCCATCAT
TC.

In some embodiments, the first nucleic acid sequence encodes bovine alpha S2 casein. In some embodiments, the first nucleic acid sequence comprises the sequence:

(SEQ ID NO: 23)
ATGAAGTTCTTCATCTTTACCTGCCTTTTGGCTGTTGCCCTTGCAAAGAATACGATGGA
ACATGTCTCCTCCAGTGAGGAATCTATCATCTCCCAGGAAACATATAAGCAGGAAAAG
AATATGGCCATTAATCCCAGCAAGGAGAACCTTTGCTCCACATTCTGCAAGGAAGTTG
TAAGGAACGCAAATGAAGAGGAATATTCTATCGGCTCATCTAGTGAGGAATCTGCTGA
AGTTGCCACTGAGGAAGTTAAGATTACTGTGGACGATAAGCACTACCAGAAAGCACT
GAATGAAATCAATCAGTTTTATCAGAAGTTCCCCCAGTATCTCCAGTATCTGTATCAAG
GTCCAATTGTTTTGAACCCATGGGATCAGGTTAAGAGGAATGCTGTTCCCATTACTCCC
ACTCTGAACAGAGAGCAGCTCTCCACCAGTGAGGAAAATTCAAAGAAGACCGTTGAC
ATGGAATCAACAGAAGTATTCACTAAGAAAACTAAACTGACTGAAGAAGAAAAGAAT
CGCCTAAATTTTCTGAAAAAAATCAGCCAGCGTTACCAGAAATTCGCCTTGCCCCAGT
ATCTCAAGACTGTTTATCAGCATCAGAAAGCTATGAAGCCATGGATTCAACCTAAGAC
AAAGGTTATTCCCTATGTGAGGTACCTTTAA.

In some embodiments, the first nucleic acid sequence encodes bovine alpha S2 casein analog.

In some embodiments, the first nucleic acid sequence comprises the sequence:

(SEQ ID NO: 29)
ATGAAGTTCTTCATCTTTACCTGCCTTTTGGCTGTTGCCCTTGCAAAGAATACGATGGA
ACATGTCTCCTCCAGTGAGGAATCTATCATCTCCCAGGAAACATATAAGCAGGAAAAG
AATATGGCCATTAATCCCAGCAAGGAGAACCTTTGCTCCACATTCTGCAAGGAAGTTG
TAAGGAACGCAAATGAAGAGGAATATTCTATCGGCTCATCTAGTGAGGAATCTGCTGA
AGTTGCCACTGAGGAAGTTAAGATTACTGTGGACGATAAGCACTACCAGAAAGCACT
GAATGAAATCAATCAGTTTTATCAGAAGTTCCCCCAGTATCTCCAGTATCTGTATCAAG
GTCCAATTGTTTTGAACCCATGGGATCAGGTTAAGAGGAATGCTGTTCCCATTACTCCC
ACTCTGAACAGAGAGCAGCTCTCCACCAGTGAGGAAAATTCAAAGAAGACCGTTGAC
ATGGAATCAACAGAAGTATTCACTAAGAAAACTAAACTGACTGAAGAAGAAAAGAAT
CGCCTAAATTTTCTGAAAAAAATCAGCCAGCGTTACCAGAAATTC.

In some embodiments, the first nucleic acid sequence encodes bovine beta casein. In some embodiments, the first nucleic acid sequence comprises the sequence:

(SEQ ID NO: 24)
ATGAAGGTCCTCATCCTTGCCTGCCTGGTGGCTCTGGCCCTTGCAAGAGAGCTGGAAG
AACTCAATGTACCTGGTGAGATTGTGGAAAGCCTTTCAAGCAGCGAGGAATCAATTAC
ACGCATCAATAAGAAAATTGAGAAGTTTCAGAGTGAGGAACAGCAGCAAACAGAGGA
TGAACTCCAGGATAAAATCCACCCCTTTGCCCAGACACAGTCTCTAGTCTATCCCTTCC
CTGGGCCCATCCCTAACAGCCTCCCACAAAACATCCCTCCTCTTACTCAAACCCCTGTG
GTGGTGCCGCCTTTCCTTCAGCCTGAAGTAATGGGAGTCTCCAAAGTGAAGGAGGCTA
TGGCCCCTAAGCACAAAGAAATGCCCTTCCCTAAATATCCAGTTGAGCCCTTTACTGA
AAGCCAGAGCCTGACTCTCACTGATGTTGAAAATCTGCACCTTCCTCTGCCTCTGCTCC
AGTCTTGGATGCACCAGCCTCACCAGCCTCTTCCTCCAACTGTCATGTTTCCTCCTCAG
TCCGTGCTGTCCCTTTCTCAGTCCAAAGTCCTGCCTGTTCCCCAGAAAGCAGTGCCCTA
TCCCCAGAGAGATATGCCCATTCAGGCCTTTCTGCTGTACCAGGAGCCTGTACTCGGTC
CTGTCCGGGGACCCTTCCCTATTATTGTCTAA.

In some embodiments, the first nucleic acid sequence encodes bovine beta casein analog. In some embodiments, the first nucleic acid sequence comprises the sequence:

(SEQ ID NO: 30)
ATGAAGGTCCTCATCCTTGCCTGCCTGGTGGCTCTGGCCCTTGCAAGAGAGCTGGAAG
AACTCAATGTACCTGGTGAGATTGTGGAAAGCCTTTCAAGCAGCGAGGAATCAATTAC
ACGCATCAATAAGAAAATTGAGAAGTTTCAGAGTGAGGAACAGCAGCAAACAGAGGA
TGAACTCCAGGATAAAATCCACCCCTTTGCCCAGACACAGTCTCTAGTCTATCCCTTCC
CTGGGCCCATCCCTAACAGCCTCCCACAAAACATCCCTCCTCTTACTCAAACCCCTGTG
GTGGTGCCGCCTTTCCTTCAGCCTGAAGTAATGGGAGTCTCCAAAGTGAAGGAGGCTA
TGGCCCCTAAGCACAAAGAAATGCCCTTCCCTAAATATCCAGTTGAGCCCTTTACTGA
AAGCCAGAGCCTGACTCTCACTGATGTTGAAAATCTGCACCTTCCTCTGCCTCTGCTCC
AGTCTTGGATGCACCAGCCTCACCAGCCTCTTCCTCCAACTGTCATGTTTCCTCCTCAG
TCCGTGCTGTCCCTTTCTCAGTCCAAAGTCCTGCCTGTTCCCCAGAAAGCAGTGCCCTA
TCCCCAGAGAGATATGCCCATTCAGGCCTTTCTGCTGTACCAGGAGCCTGTACTCGGTC
CTGTCCGGGGACCCTTC.

In some embodiments, the first nucleic acid sequence encodes bovine beta lactoglobulin. In some embodiments, the first nucleic acid sequence comprises the sequence:

(SEQ ID NO: 25)
ATGAAGTGCCTCCTGCTTGCCCTGGCCCTCACTTGTGGCGCCCAGGCCCTCATTGTCAC
CCAGACCATGAAGGGCCTGGATATCCAGAAGGTGGCGGGGACTTGGTACTCCTTGGCC
ATGGCGGCCAGCGACATCTCCCTGCTGGACGCCCAGAGTGCCCCCCTGAGAGTGTATG
TGGAGGAGCTGAAGCCCACCCCTGAGGGCGACCTGGAGATCCTGCTGCAGAAATGGG
AGAACGGTGAGTGTGCTCAGAAGAAGATCATTGCAGAAAAAACCAAGATCCCTGCGG
TGTTCAAGATCGATGCCTTGAATGAGAACAAAGTCCTTGTGCTGGACACCGACTACAA
AAAGTACCTGCTCTTCTGCATGGAGAACAGTGCTGAGCCCGAGCAAAGCCTGGCCTGC
CAGTGCCTGGTCAGGACCCCGGAGGTGGACGACGAGGCCCTGGAGAAATTCGACAAA
GCCCTCAAGGCCCTGCCCATGCACATCCGGCTGTCCTTCAACCCAACCCAGCTGGAGG
AGCAGTGCCACATC.

In some embodiments, the first nucleic acid sequence encodes bovine beta lactoglobulin analog. In some embodiments, the first nucleic acid sequence comprises the sequence:

(SEQ ID NO: 31)
ATGAAGTGCCTCCTGCTTGCCCTGGCCCTCACTTGTGGCGCCCAGGCCCTCATTGTCAC
CCAGACCATGAAGGGCCTGGATATCCAGAAGGTGGCGGGGACTTGGTACTCCTTGGCC
ATGGCGGCCAGCGACATCTCCCTGCTGGACGCCCAGAGTGCCCCCCTGAGAGTGTATG
TGGAGGAGCTGAAGCCCACCCCTGAGGGCGACCTGGAGATCCTGCTGCAGAAATGGG
AGAACGGTGAGTGTGCTCAGAAGAAGATCATTGCAGAAAAAACCAAGATCCCTGCGG
TGTTCAAGATCGATGCCTTGAATGAGAACAAAGTCCTTGTGCTGGACACCGACTACAA
AAAGTACCTGCTCTTCTGCATGGAGAACAGTGCTGAGCCCGAGCAAAGCCTGGCCTGC
CAGTGCCTGGTCAGGACCCCGGAGGTGGACGACGAGGCCCTGGAGAAATTCGACAAA
GCCCTCAAGGCCCTGCCCATGCACATCCGGCTGTCCTTC.

In some embodiments, the first nucleic acid sequence encodes fungal beta lactoglobulin. In some embodiments, the first nucleic acid sequence comprises the sequence:

(SEQ ID NO: 26)
ATGAAGTTTTTCGCCATTGCCGCCCTATTTGCCGCCGCTGCCGTTGCCCAGCCTAACGT
CATCTCGAAGCGGCTCATCGTCACCCAGACCATGAAGGGCCTCGACATCCAGAAGGTC
GCCGGCACCTGGTACAGCCTCGCCATGGCCGCCAGCGACATCAGCCTGCTCGACGCCC
AGAGCGCCCCTCTCCGCGTCTACGTCGAGGAGCTGAAGCCCACGCCTGAGGGCGACCT
CGAGATCCTCCTGCAGAAGTGGGAGAACGGCGAGTGCGCCCAGAAGAAGATCATTGC
CGAGAAGACGAAGATCCCCGCCGTGTTCAAGATCGACGCCCTCAACGAGAACAAGGT
CCTCGTCCTCGACACCGACTACAAGAAGTACCTCCTGTTCTGCATGGAGAACTCCGCC
GAGCCTGAGCAGAGCCTCGCCTGCCAGTGCCTCGTTCGCACGCCCGAGGTCGACGACG
AGGCCCTCGAGAAGTTCGACAAGGCCCTCAAGGCTCTCCCCATGCACATCCGCCTCAG
CTTCAACCCCACGCAGCTCGAGGAGCAGTGCCACATC.

In some embodiments, the first nucleic acid sequence encodes fungal beta lactoglobulin analog. In some embodiments, the first nucleic acid sequence comprises the sequence:

(SEQ ID NO: 32)
ATGAAGTTTTTCGCCATTGCCGCCCTATTTGCCGCCGCTGCCGTTGCCCAGCCTAACGT
CATCTCGAAGCGGCTCATCGTCACCCAGACCATGAAGGGCCTCGACATCCAGAAGGTC
GCCGGCACCTGGTACAGCCTCGCCATGGCCGCCAGCGACATCAGCCTGCTCGACGCCC
AGAGCGCCCCTCTCCGCGTCTACGTCGAGGAGCTGAAGCCCACGCCTGAGGGCGACCT
CGAGATCCTCCTGCAGAAGTGGGAGAACGGCGAGTGCGCCCAGAAGAAGATCATTGC
CGAGAAGACGAAGATCCCCGCCGTGTTCAAGATCGACGCCCTCAACGAGAACAAGGT
CCTCGTCCTCGACACCGACTACAAGAAGTACCTCCTGTTCTGCATGGAGAACTCCGCC
GAGCCTGAGCAGAGCCTCGCCTGCCAGTGCCTCGTTCGCACGCCCGAGGTCGACGACG
AGGCCCTCGAGAAGTTCGACAAGGCCCTCAAGGCTCTCCCCATGCACATCCGCCTCAG
CTTC.

In some embodiments, the first nucleic acid encodes a bovine beta lactoglobulin devoid of a signal peptide. In some embodiments, the first nucleic acid encodes a signal peptide from a fungus and a bovine beta lactoglobulin devoid of a signal peptide. In some embodiments, the fungus is aspergillus oryzae. In some embodiments, bovine beta lactoglobulin devoid of a signal peptide comprises the sequence:

(SEQ ID NO: 43)
CTCATCGTCACCCAGACCATGAAGGGCCTCGACATCCAGAAGGTCGCCGGCACCTGGT
ACAGCCTCGCCATGGCCGCCAGCGACATCAGCCTGCTCGACGCCCAGAGCGCCCCTCT
CCGCGTCTACGTCGAGGAGCTGAAGCCCACGCCTGAGGGCGACCTCGAGATCCTCCTG
CAGAAGTGGGAGAACGGCGAGTGCGCCCAGAAGAAGATCATTGCCGAGAAGACGAA
GATCCCCGCCGTGTTCAAGATCGACGCCCTCAACGAGAACAAGGTCCTCGTCCTCGAC
ACCGACTACAAGAAGTACCTCCTGTTCTGCATGGAGAACTCCGCCGAGCCTGAGCAGA
GCCTCGCCTGCCAGTGCCTCGTTCGCACGCCCGAGGTCGACGACGAGGCCCTCGAGAA
GTTCGACAAGGCCCTCAAGGCTCTCCCCATGCACATCCGCCTCAGCTTCAACCCCACG
CAGCTCGAGGAGCAGTGCCACATC.

In some embodiments, bovine beta lactoglobulin devoid of a signal peptide comprises the sequence:

(SEQ ID NO: 44)
CTCATCGTCACCCAGACCATGAAGGGCCTCGACATCCAGAAGGTCGCCGGCAC
CTGGTACAGCCTCGCCATGGCCGCCAGCGACATCAGCCTGCTCGACGCCCAGAGCGCC
CCTCTCCGCGTCTACGTCGAGGAGCTGAAGCCCACGCCTGAGGGCGACCTCGAGATCC
TCCTGCAGAAGTGGGAGAACGGCGAGTGCGCCCAGAAGAAGATCATTGCCGAGAAGA
CGAAGATCCCCGCCGTGTTCAAGATCGACGCCCTCAACGAGAACAAGGTCCTCGTCCT
CGACACCGACTACAAGAAGTACCTCCTGTTCTGCATGGAGAACTCCGCCGAGCCTGAG
CAGAGCCTCGCCTGCCAGTGCCTCGTTCGCACGCCCGAGGTCGACGACGAGGCCCTCG
AGAAGTTCGACAAGGCCCTCAAGGCTCTCCCCATGCACATCCGCCTCAGCTTC.

In some embodiments, the signal peptide comprises the sequence:

(SEQ ID NO: 45)
ATGAAGTTTTTCGCCATTGCCGCCCTATTTGCCGCCGCTGCCGTTGCCC
AGCCTAACGTCATCTCGAAGCGG.

In some embodiments, the first nucleic acid sequence encodes bovine alpha lactalbumin. In some embodiments, the first nucleic acid sequence comprises the sequence:

(SEQ ID NO: 27)
ATGATGTCCTTTGTCTCTCTGCTCCTGGTAGGCATCCTATTCCATGCCACCCAGGCTGA
ACAGTTAACAAAATGTGAGGTGTTCCGGGAGCTGAAAGACTTGAAGGGCTACGGAGG
TGTCAGTTTGCCTGAATGGGTCTGTACCACGTTTCATACCAGTGGTTATGACACACAAG
CCATAGTACAAAACAATGACAGCACAGAATATGGACTCTTCCAGATAAATAATAAAA
TTTGGTGCAAAGACGACCAGAACCCTCACTCAAGCAACATCTGTAACATCTCCTGTGA
CAAGTTCCTGGATGATGATCTTACTGATGACATTATGTGTGTCAAGAAGATTCTGGAT
AAAGTAGGAATTAACTACTGGTTGGCCCATAAAGCACTCTGTTCTGAGAAGCTGGATC
AGTGGCTCTGTGAGAAGTTGTGA.

In some embodiments, the first nucleic acid sequence encodes bovine alpha lactalbumin analog. In some embodiments, the first nucleic acid sequence comprises the sequence:

(SEQ ID NO: 33)
ATGATGTCCTTTGTCTCTCTGCTCCTGGTAGGCATCCTATTCCAT
GCCACCCAGGCTGAACAGTTAACAAAATGTGAGGTGTTCCGGGAG
CTGAAAGACTTGAAGGGCTACGGAGGTGTCAGTTTGCCTGAATGG
GTCTGTACCACGTTTCATACCAGTGGTTATGACACACAAGCCATA
GTACAAAACAATGACAGCACAGAATATGGACTCTTCCAGATAAAT
AATAAAATTTGGTGCAAAGACGACCAGAACCCTCACTCAAGCAAC
ATCTGTAACATCTCCTGTGACAAGTTC.

In some embodiments, the first nucleotide encodes a functional analog of any one of SEQ ID Nos: 22-33, having at least 80%, 90%, 95%, 99% identity or homology thereto, or any value and range therebetween. Each possibility represents a separate embodiment of the invention.

As used herein, the term “functional analog” refers to any polypeptide characterized by having at least 60%, 70%, 80%, 90% or 95% the activity or function of a polypeptide as disclosed herein.

In some embodiments, the second nucleic acid comprises nucleotides 376-381 within the sequence encoding full length kapa casein. In some embodiments, the second nucleic acid comprises the sequence set forth in SEQ ID NO: 34. In some embodiments, the second nucleic acid comprises the sequence: TTNATG, wherein N is A, T, C or G. In some embodiments, the second nucleic acid comprises the sequence: TTTATG.

In some embodiments, the third nucleic acid encodes kappa casein macropeptide. In some embodiments, the third nucleic sequence comprises the sequence: ATGGCCATTCCACCAAAGAAAAATCAGGATAAAACAGAAATCCCTACCATCAATACC ATTGCTAGTGGTGAGCCTACAAGTACACCTACCACCGAAGCAGTAGAGAGCACTGTAG CTACTCTAGAAGATTCTCCAGAAGTTATTGAGAGCCCACCTGAGATCAACACAGTCCA AGTTACTTCAACTGCAGTC (SEQ ID NO: 35). In some embodiments, the third nucleic acid encodes kappa casein macropeptide or a functional analog thereof. In some embodiments, the third nucleic sequence the comprises sequence:

(SEQ ID NO: 36)
GCCATTCCACCAAAGAAAAATCAGGATAAAACAGAAATCCCTACC
ATCAATACCATTGCTAGTGGTGAGCCTACAAGTACACCTACCACC
GAAGCAGTAGAGAGCACTGTAGCTACTCTAGAAGATTCTCCAGAA
GTTATTGAGAGCCCACCTGAGATCAACACAGTCCAAGTTACTTCA
ACTGCAGTC.

In some embodiments, the third nucleic acids encodes a functional analog of SEQ ID NO: 35 or SEQ ID NO: 36, having at least 80%, 90%, 95%, 99% identity or homology thereto, or any value and range therebetween. Each possibility represents a separate embodiment of the invention.

In some embodiments, the chimeric polynucleotide of the invention comprises the sequence: ATGAAACTTCTCATCCTTACCTGTCTTGTGGCTGTTGCTCTTGCTAGGCCTAAACATCC TATCAAGCACCAAGGACTCCCTCAAGAAGTCCTCAATGAAAATTTACTCAGGTTTTTT GTGGCACCTTTTCCAGAAGTGTTTGGAAAGGAGAAGGTCAATGAACTGAGCAAGGAT ATTGGGAGTGAATCAACTGAGGATCAAGCCATGGAAGATATTAAGCAAATGGAAGCT GAAAGCATTTCGTCAAGTGAGGAAATTGTTCCCAATAGTGTTGAGCAGAAGCACATTC AAAAGGAAGATGTGCCCTCTGAGCGTTACCTGGGTTATCTGGAACAGCTTCTCAGACT GAAAAAATACAAAGTACCCCAGCTGGAAATTGTTCCCAATAGTGCTGAGGAACGACTT CACAGTATGAAAGAGGGAATCCATGCCCAACAGAAAGAACCTATGATAGGAGTGAAT CAGGAACTGGCCTACTTCTACCCTGAGCTTTTCAGACAATTCTACCAGCTGGATGCCTA TCCATCTGGTGCCTGGTATTACGTTCCACTAGGCACACAATACACTGATGCCCCATCAT TCATGGCCATTCCACCAAAGAAAAATCAGGATAAAACAGAAATCCCTACCATCAATAC CATTGCTAGTGGTGAGCCTACAAGTACACCTACCACCGAAGCAGTAGAGAGCACTGTA GCTACTCTAGAAGATTCTCCAGAAGTTATTGAGAGCCCACCTGAGATCAACACAGTCC AAGTTACTTCAACTGCAGTC (SEQ ID NO: 37), or a functional analog thereof, having at least 80%, 90%, 95%, 99% identity or homology thereto, or any value and range therebetween. Each possibility represents a separate embodiment of the invention.

In some embodiments, the chimeric polynucleotide of the invention comprises the sequence: ATGAAGTTCTTCATCTTTACCTGCCTTTTGGCTGTTGCCCTTGCAAAGAATAC GATGGAACATGTCTCCTCCAGTGAGGAATCTATCATCTCCCAGGAAACATATAAGCAG GAAAAGAATATGGCCATTAATCCCAGCAAGGAGAACCTTTGCTCCACATTCTGCAAGG AAGTTGTAAGGAACGCAAATGAAGAGGAATATTCTATCGGCTCATCTAGTGAGGAATC TGCTGAAGTTGCCACTGAGGAAGTTAAGATTACTGTGGACGATAAGCACTACCAGAAA GCACTGAATGAAATCAATCAGTTTTATCAGAAGTTCCCCCAGTATCTCCAGTATCTGTA TCAAGGTCCAATTGTTTTGAACCCATGGGATCAGGTTAAGAGGAATGCTGTTCCCATT ACTCCCACTCTGAACAGAGAGCAGCTCTCCACCAGTGAGGAAAATTCAAAGAAGACC GTTGACATGGAATCAACAGAAGTATTCACTAAGAAAACTAAACTGACTGAAGAAGAA AAGAATCGCCTAAATTTTCTGAAAAAAATCAGCCAGCGTTACCAGAAATTCATGGCCA TTCCACCAAAGAAAAATCAGGATAAAACAGAAATCCCTACCATCAATACCATTGCTAG TGGTGAGCCTACAAGTACACCTACCACCGAAGCAGTAGAGAGCACTGTAGCTACTCTA GAAGATTCTCCAGAAGTTATTGAGAGCCCACCTGAGATCAACACAGTCCAAGTTACTT CAACTGCAGTC (SEQ ID NO: 38) , or a functional analog thereof, having at least 80%, 90%, 95%, 99% identity or homology thereto, or any value and range therebetween. Each possibility represents a separate embodiment of the invention.

In some embodiments, the chimeric polynucleotide of the invention comprises the sequence: ATGAAGGTCCTCATCCTTGCCTGCCTGGTGGCTCTGGCCCTTGCAAGAGAGCT GGAAGAACTCAATGTACCTGGTGAGATTGTGGAAAGCCTTTCAAGCAGCGAGGAATC AATTACACGCATCAATAAGAAAATTGAGAAGTTTCAGAGTGAGGAACAGCAGCAAAC AGAGGATGAACTCCAGGATAAAATCCACCCCTTTGCCCAGACACAGTCTCTAGTCTAT CCCTTCCCTGGGCCCATCCCTAACAGCCTCCCACAAAACATCCCTCCTCTTACTCAAAC CCCTGTGGTGGTGCCGCCTTTCCTTCAGCCTGAAGTAATGGGAGTCTCCAAAGTGAAG GAGGCTATGGCCCCTAAGCACAAAGAAATGCCCTTCCCTAAATATCCAGTTGAGCCCT TTACTGAAAGCCAGAGCCTGACTCTCACTGATGTTGAAAATCTGCACCTTCCTCTGCCT CTGCTCCAGTCTTGGATGCACCAGCCTCACCAGCCTCTTCCTCCAACTGTCATGTTTCC TCCTCAGTCCGTGCTGTCCCTTTCTCAGTCCAAAGTCCTGCCTGTTCCCCAGAAAGCAG TGCCCTATCCCCAGAGAGATATGCCCATTCAGGCCTTTCTGCTGTACCAGGAGCCTGTA CTCGGTCCTGTCCGGGGACCCTTCATGGCCATTCCACCAAAGAAAAATCAGGATAAAA CAGAAATCCCTACCATCAATACCATTGCTAGTGGTGAGCCTACAAGTACACCTACCAC CGAAGCAGTAGAGAGCACTGTAGCTACTCTAGAAGATTCTCCAGAAGTTATTGAGAGC CCACCTGAGATCAACACAGTCCAAGTTACTTCAACTGCAGTC (SEQ ID NO: 39), or a functional analog thereof, having at least 80%, 90%, 95%, 99% identity or homology thereto, or any value and range therebetween. Each possibility represents a separate embodiment of the invention.

In some embodiments, the chimeric polynucleotide of the invention comprises the sequence: ATGAAGTGCCTCCTGCTTGCCCTGGCCCTCACTTGTGGCGCCCAGGCCCTCATTGTCAC CCAGACCATGAAGGGCCTGGATATCCAGAAGGTGGCGGGGACTTGGTACTCCTTGGCC ATGGCGGCCAGCGACATCTCCCTGCTGGACGCCCAGAGTGCCCCCCTGAGAGTGTATG TGGAGGAGCTGAAGCCCACCCCTGAGGGCGACCTGGAGATCCTGCTGCAGAAATGGG AGAACGGTGAGTGTGCTCAGAAGAAGATCATTGCAGAAAAAACCAAGATCCCTGCGG TGTTCAAGATCGATGCCTTGAATGAGAACAAAGTCCTTGTGCTGGACACCGACTACAA AAAGTACCTGCTCTTCTGCATGGAGAACAGTGCTGAGCCCGAGCAAAGCCTGGCCTGC CAGTGCCTGGTCAGGACCCCGGAGGTGGACGACGAGGCCCTGGAGAAATTCGACAAA GCCCTCAAGGCCCTGCCCATGCACATCCGGCTGTCCTTCATGGCCATTCCACCAAAGA AAAATCAGGATAAAACAGAAATCCCTACCATCAATACCATTGCTAGTGGTGAGCCTAC AAGTACACCTACCACCGAAGCAGTAGAGAGCACTGTAGCTACTCTAGAAGATTCTCCA GAAGTTATTGAGAGCCCACCTGAGATCAACACAGTCCAAGTTACTTCAACTGCAGTC (SEQ ID NO: 40), or a functional analog thereof, having at least 80%, 90%, 95%, 99% identity or homology thereto, or any value and range therebetween. Each possibility represents a separate embodiment of the invention.

In some embodiments, the chimeric polynucleotide of the invention comprises the sequence: ATGAAGTTTTTCGCCATTGCCGCCCTATTTGCCGCCGCTGCCGTTGCCCAGCCTAACGT CATCTCGAAGCGGCTCATCGTCACCCAGACCATGAAGGGCCTCGACATCCAGAAGGTC GCCGGCACCTGGTACAGCCTCGCCATGGCCGCCAGCGACATCAGCCTGCTCGACGCCC AGAGCGCCCCTCTCCGCGTCTACGTCGAGGAGCTGAAGCCCACGCCTGAGGGCGACCT CGAGATCCTCCTGCAGAAGTGGGAGAACGGCGAGTGCGCCCAGAAGAAGATCATTGC CGAGAAGACGAAGATCCCCGCCGTGTTCAAGATCGACGCCCTCAACGAGAACAAGGT CCTCGTCCTCGACACCGACTACAAGAAGTACCTCCTGTTCTGCATGGAGAACTCCGCC GAGCCTGAGCAGAGCCTCGCCTGCCAGTGCCTCGTTCGCACGCCCGAGGTCGACGACG AGGCCCTCGAGAAGTTCGACAAGGCCCTCAAGGCTCTCCCCATGCACATCCGCCTCAG CTTCATGGCCATCCCCCCCAAGAAGAACCAGGACAAGACCGAGATCCCCACCATCAAC ACCATCGCCTCCGGCGAGCCCACCTCCACCCCCACCACCGAGGCCGTCGAGTCCACCG TCGCCACCCTGGAGGACTCCCCCGAGGTCATCGAGTCCCCCCCCGAGATCAACACCGT CCAGGTCACCTCCACCGCCGTC (SEQ ID NO: 41), or a functional analog thereof, having at least 80%, 90%, 95%, 99% identity or homology thereto, or any value and range therebetween. Each possibility represents a separate embodiment of the invention.

In some embodiments, the chimeric polynucleotide of the invention comprises the sequence: ATGATGTCCTTTGTCTCTCTGCTCCTGGTAGGCATCCTATTCCATGCCACCCAGGCTGA ACAGTTAACAAAATGTGAGGTGTTCCGGGAGCTGAAAGACTTGAAGGGCTACGGAGG TGTCAGTTTGCCTGAATGGGTCTGTACCACGTTTCATACCAGTGGTTATGACACACAAG CCATAGTACAAAACAATGACAGCACAGAATATGGACTCTTCCAGATAAATAATAAAA TTTGGTGCAAAGACGACCAGAACCCTCACTCAAGCAACATCTGTAACATCTCCTGTGA CAAGTTCATGGCCATTCCACCAAAGAAAAATCAGGATAAAACAGAAATCCCTACCATC AATACCATTGCTAGTGGTGAGCCTACAAGTACACCTACCACCGAAGCAGTAGAGAGC ACTGTAGCTACTCTAGAAGATTCTCCAGAAGTTATTGAGAGCCCACCTGAGATCAACA CAGTCCAAGTTACTTCAACTGCAGTC (SEQ ID NO: 42), or a functional analog thereof, having at least 80%, 90%, 95%, 99% identity or homology thereto, or any value and range therebetween. Each possibility represents a separate embodiment of the invention.

As used herein, the term “analog” refers to a polynucleotide that is similar, but not identical, to the polynucleotide as disclosed herein, that encodes a polypeptide that is still capable of forming casein and/or casein-like micelles. An analog may have deletions or mutations that result in an amino acids sequence that is different than the amino acid sequence encoded by the polynucleotide of the invention. It should be understood that all analogs of the polynucleotide of the invention would still be capable of encoding a chimeric polypeptide forming casein and/or casein-like micelles. Further, an analog may be analogous to a fragment of the polynucleotide of the invention, however, in such a case the fragment must comprise at least 100, 200, or 400 consecutive nucleotides of the chimeric polynucleotide of the invention.

As used herein, the term “functional analog” refers to any nucleic acid sequence encoding a polypeptide as disclosed herein, wherein at least 80%, 90%, 95%, 99%, or any value and range therebetween, of the activity, functionality, or both, of the disclosed polypeptide herein is preserved. Each possibility represents a separate embodiment of the invention.

In some embodiments, the functional analog comprises or is characterized by having at least 80%, 90%, 95%, 99%, or any value and range therebetween, homology or identity to one sequence selected from: SEQ ID Nos: 37-42. Each possibility represents a separate embodiment of the invention.

In some embodiments, the chimeric polynucleotide further comprises one or more nucleic acid sequences encoding: a tag, a linker, or a combination thereof.

In some embodiments, the tag may be suitable for protein isolation and/or purification. In some embodiments, the tag comprises a poly histidine tag. In some embodiments, the tag comprises S-tag.

In some embodiments, the linker is a flexible linker.

### Plasmids and Expression Vectors

According to some embodiments, there is provided a plasmid comprising the chimeric polynucleotide disclosed herein.

In some embodiments, the plasmid is an expression vector.

Expressing of a polynucleotide within a cell is well known to one skilled in the art. It can be carried out by, among many methods, transfection, viral infection, or direct alteration of the cell's genome. In some embodiments, the polynucleotide is in an expression vector such as plasmid or viral vector.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector, a virgaviridae viral vector, or a poxviral vector. The barley stripe mosaic virus (BSMV), the tobacco rattle virus and the cabbage leaf curl geminivirus (CbLCV) may also be used. The promoters may be active in plant cells. The promoters may be a viral promoter.

In some embodiments, the polynucleotide as disclosed herein is operably linked to a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell). In some embodiments, the promoter is operably linked to the polynucleotide of the invention. In some embodiments, the promoter is a heterologous promoter. In some embodiments, the promoter is the endogenous promoter.

In some embodiments, the vector is introduced into the cell by standard methods including electroporation (e.g., as described in From et al., Proc. Natl. Acad. Sci. USA 82, 5824 (1985)), heat shock, infection by viral vectors, high velocity ballistic penetration by small particles with the nucleic acid either within the matrix of small beads or particles, or on the surface (Klein et al., Nature 327. 70-73 (1987)), such as biolistic use of coated particles, and needle-like particles, Agrobacterium Ti plasmids and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. The promoter may extend upstream or downstream of the transcriptional start site and may be any size ranging from a few base pairs to several kilo-bases.

In some embodiments, the polynucleotide is transcribed by RNA polymerase II (RNAP II and Pol II). RNAP II is an enzyme found in eukaryotic cells, known to catalyze the transcription of DNA to synthesize precursors of mRNA and most snRNA and microRNA.

In some embodiments, a plant expression vector is used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

In some embodiments, expression vectors containing regulatory clements from cukaryotic viruses such as retroviruses are used by the present invention. SV40 vectors include pSVT7 and pMT2. In some embodiments, vectors derived from bovine papilloma virus include pBV-IMTHA, and vectors derived from Epstein Bar virus include pHEBO, and p205. Other exemplary vectors include pMSG, pAV009/A+, pMTO10/A+, pMAMnco-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in cukaryotic cells.

In some embodiments, recombinant viral vectors, which offer advantages such as systemic infection and targeting specificity, are used for in vivo expression. In one embodiment, systemic infection is inherent in the life cycle of, for example, the retrovirus and is the process by which a single infected cell produces many progeny virions that infect neighboring cells. In one embodiment, the result is that a large area becomes rapidly infected, most of which was not initially infected by the original viral particles. In one embodiment, viral vectors are produced that are unable to spread systemically. In one embodiment, this characteristic can be useful if the desired purpose is to introduce a specified gene into only a localized number of targeted cells.

In some embodiments, plant viral vectors are used. In some embodiments, a wild-type virus is used. In some embodiments, a deconstructed virus such as are known in the art is used. In some embodiments, Agrobacterium is used to introduce the vector of the invention into a virus.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, agrobacterium Ti plasmids and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield, or activity of the expressed polypeptide.

In some embodiments, the plasmid or expression vector comprises the nucleic acid sequences set forth in one of SEQ ID Nos: 37-42.

### Chimeric Polypeptides

According to some embodiments, there is provided a chimeric polypeptide encoded, expressed, or both from: the chimeric polynucleotide disclosed herein; or the plasmid disclosed herein.

In some embodiments, the chimeric polypeptide of the invention comprises the amino acid sequence:

(SEQ ID NO: 16)
MKLLILTCLVAVALARPKHPIKHQGLPQEVLNENLLRFFVAPFPE
VFGKEKVNELSKDIGSESTEDQAMEDIKQMEAESISSSEEIVPNS
VEQKHIQKEDVPSERYLGYLEQLLRLKKYKVPQLEIVPNSAEERL
HSMKEGIHAQQKEPMIGVNQELAYFYPELFRQFYQLDAYPSGAWY
YVPLGTQYTDAPSFMAIPPKKNQDKTEIPTINTIASGEPTSTPTT
EAVESTVATLEDSPEVIESPPEINTVQVTSTAV.

In some embodiments, the chimeric polypeptide of the invention comprises the amino acid sequence:

(SEQ ID NO: 17)
MKFFIFTCLLAVALAKNTMEHVSSSEESIISQETYKQEKNMAINP
SKENLCSTFCKEVVRNANEEEYSIGSSSEESAEVATEEVKITVDD
KHYQKALNEINQFYQKFPQYLQYLYQGPIVLNPWDQVKRNAVPIT
PTLNREQLSTSEENSKKTVDMESTEVFTKKTKLTEEEKNRLNFLK
KISQRYQKFMAIPPKKNQDKTEIPTINTIASGEPTSTPTTEAVES
TVATLEDSPEVIESPPEINTVQVTSTAV.

In some embodiments, the chimeric polypeptide of the invention comprises the amino acid sequence:

(SEQ ID NO: 18)
MKVLILACLVALALARELEELNVPGEIVESLSSSEESITRINKKI
EKFQSEEQQQTEDELQDKIHPFAQTQSLVYPFPGPIPNSLPQNIP
PLTQTPVVVPPFLQPEVMGVSKVKEAMAPKHKEMPFPKYPVEPFT
ESQSLTLTDVENLHLPLPLLQSWMHQPHQPLPPTVMFPPQSVLSL
SQSKVLPVPQKAVPYPQRDMPIQAFLLYQEPVLGPVRGPFMAIPP
KKNQDKTEIPTINTIASGEPTSTPTTEAVESTVATLEDSPEVIES
PPEINTVQVTSTAV.

In some embodiments, the chimeric polypeptide of the invention comprises the amino acid sequence:

(SEQ ID NO: 19)
MKCLLLALALTCGAQALIVTQTMKGLDIQKVAGTWYSLAMAASDI
SLLDAQSAPLRVYVEELKPTPEGDLEILLQKWENGECAQKKIIAE
KTKIPAVFKIDALNENKVLVLDTDYKKYLLFCMENSAEPEQSLAC
QCLVRTPEVDDEALEKFDKALKALPMHIRLSFMAIPPKKNQDKTE
IPTINTIASGEPTSTPTTEAVESTVATLEDSPEVIESPPEINTVQ
VTSTAV.

In some embodiments, the chimeric polypeptide of the invention comprises the amino acid sequence:

(SEQ ID NO: 20)
MKFFAIAALFAAAAVAQPNVISKRLIVTQTMKGLDIQKVAGTWYS
LAMAASDISLLDAQSAPLRVYVEELKPTPEGDLEILLQKWENGEC
AQKKIIAEKTKIPAVFKIDALNENKVLVLDTDYKKYLLFCMENSA
EPEQSLACQCLVRTPEVDDEALEKFDKALKALPMHIRLSFMAIPP
KKNQDKTEIPTINTIASGEPTSTPTTEAVESTVATLEDSPEVIES
PPEINTVQVTSTAV.

In some embodiments, the chimeric polypeptide of the invention comprises the amino acid sequence:

(SEQ ID NO: 21)
MMSFVSLLLVGILFHATQAEQLTKCEVFRELKDLKGYGGVSLPEW
VCTTFHTSGYDTQAIVQNNDSTEYGLFQINNKIWCKDDQNPHSSN
ICNISCDKFMAIPPKKNQDKTEIPTINTIASGEPTSTPTTEAVES
TVATLEDSPEVIESPPEINTVQVTSTAV.

As used herein, the terms “peptide”, “polypeptide” and “protein” are interchangeable and refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides, polypeptides, and proteins described have modifications rendering them more stable while in the organism or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

### Cells

According to some embodiments, there is provided a cell, comprising: (a) the chimeric polynucleotide disclosed herein; (b) the plasmid disclosed herein; (c) the chimeric polypeptide disclosed herein; or (d) any combination of (a) to (c).

In some embodiments, the cell comprises any portion, fraction, organelle, or any combination thereof, of a cell.

In some embodiments, one of: the chimeric polynucleotide disclosed herein, the plasmid disclosed herein, the chimeric polypeptide disclosed herein, or any combination thereof, is expressed in a transgenic or a transfected cell. In some embodiments the transfected cell is a primary cell. In some embodiments the transfected cell is a stem cell. In some embodiments, the transfected cell is derived from a cell line.

In some embodiments, the cell is transfected by a transient transfection. In some embodiments, the cell is transfected by stable transfection.

As used herein, the words “transfection”, “transduction”, and “transformation” are interchangeable.

The term “cell line”, as used herein, refers to a defined population of cells that can be maintained in culture for an extended period of time, retaining stability of certain phenotypes and functions. A person of ordinary skill in the art will recognize cell lines that can be transfected for recombinant protein, or polypeptide, expression, such as but not limited to: human embryonic kidney 293 cell line, and Chinese hamster ovary (CHO) cell line, Baby hamster kidney fibroblasts (BHK21), NSO cell line from murine myeloma, and SP2/O-Ag14 cell line.

In some embodiments the cell is a eukaryotic cell. In some embodiments the cell is a mammalian cell. In some embodiments the cell is an epithelial cell. In some embodiments the cell is derived from a mammary gland. In some embodiments, the cell is derived from a rodent.

In some embodiments the cell is a yeast. In some embodiments the cell is Pichia pastoris.

In some embodiments, the cell is prokaryote cell. In some embodiments, the cell comprises a bacterium or a bacterial cell.

### Compositions

According to some embodiments, there is provided a composition comprising: (a) the chimeric polynucleotide disclosed herein; (b) the plasmid disclosed herein; (c) the chimeric polypeptide disclosed herein; (d) the cell or any portion thereof as disclosed herein; or (c) any combination of (a) to (d); and an acceptable carrier.

In some embodiments, the carrier is an edible carrier. In some embodiments, the carrier is a dairy substituent. Examples for dairy substituents can be, but not limited to: a milk, a yogurt, a cheese, a butter, a caseinate, a cream, an infant formula, an ice cream, a frozen custard, a cottage cheese, a cream cheese, a crème fraiche, and a curd.

In some embodiments, the carrier is an aquas solution. In some embodiments, the composition comprises the polypeptide of the invention without, or prior, processing by the method disclosed herein. In some embodiments, the composition comprises the polypeptide of the invention post processing by the method disclosed herein.

In some embodiments, the composition disclosed herein is a dairy substituent. In some embodiments, the composition disclosed herein, and the method disclosed herein, can include using one or more ingredients from a group comprising: ash, whey proteins, lipids, flavor compounds, color balancing agents, and sweetening agents.

In some embodiments, the composition described herein, or the method described herein, comprises the use of ash, or minerals salts. Ash can, e.g., include one or more (two, three, four, five, six, seven, eight, nine, ten, eleven, twelve, thirteen, fourteen fifteen, sixteen, seventeen, eighteen, nineteen, or twenty) of: calcium, phosphorous, potassium, sodium, citrate, chloride, phosphate, magnesium, iron, molybdenum, manganese, copper, thiamin (vitamin B1), riboflavin (vitamin B2), niacin (vitamin B3), pantothenic acid (vitamin B5), vitamin B6 (pyridoxine), vitamin B12 (cobalamin), vitamin C, folate, vitamins A, vitamin D, vitamin E, and vitamin K. In some examples, the ash includes one or more (two or three) of CaC12, KH2PO4, and Na3 citrate. Ash can be provided as a powder or as a solution. In some embodiments, total ash concentration of the composition is within the range 0.1-10% (w/w).

As used herein, “ash” is the inorganic residue from the incineration of organic matter. Ash refers to additional components, and aspects of ash are known in the art.

In some embodiments, the composition comprises whey proteins, comprising: β-lactoglobulin, α-lactalbumin, serum albumin, immunoglobulins, lactoferrin, and transferrin.

In some embodiments, the composition comprises one or more lipids. In some embodiments, the composition comprises: one or more fats, one or more oils, one or more monoglycerides, diglycerides, and/or triglycerides, one or more free fatty acids, and one or more phospholipids. Exemplary oils, monoglycerides, diglycerides, free fatty acids, and phospholipids are described below. Additional examples of fats, oils, monoglycerides, diglycerides, triglycerides, free fatty acids, and phospholipids are known in the art.

Oils used in the present compositions or methods can include, e.g., plant-derived oils. Non-limiting examples of plant-based oils include sunflower oil, coconut oil, peanut oil, corn oil, cottonseed oil, olive oil, palm oil, rapeseed oil, safflower oil, sesame oil, soybean oil, almond oil, beech nut oil, Brazil nut oil, cashew oil, hazelnut oil, macadamia nut oil, mongongo nut oil, pecan oil, pine nut oil, pistachio nut oil, walnut oil, and avocado oil.

Monoglycerides and diglycerides that can be used in the present invention can be plant-derived monoglycerides and diglycerides. For example, monoglycerides and diglycerides can be derived from sunflowers, coconuts, peanuts, cottonseed, olives, palm, rapeseed, safflowers, sesame seed, soybeans, almonds, beech nuts, Brazil nuts, cashews, hazelnuts, macadamia nuts, mongongo nuts, pecans, pine nuts, pistachios, walnuts, and avocados. The monoglycerides and diglycerides can include the acyl chain of any of the free fatty acids listed herein. Additional examples of monoglycerides and diglycerides are known in the art.

The composition can include the use of one or more free fatty acids. Non-limiting examples of free fatty acids include butyric acid, caproic acid, caprylic acid, and capric acid. Additional examples of fatty acids include lauric acid, myristic acid, palmitic acid, stearic acid, arachidic acid, behenic acid, lignoceric acid, cerotic acid, myristoleic acid, pamitoleic acid, sapienic acid, oleic acid, claidic acid, vaccenic acid, linoleic acid, linoelaidic acid, α-linolenic acid, arachidonic acid, cicosapentaenoic acid, crucic acid, docosahexaenoic acid, omega-3 fatty acids, and omega-6 fatty acids. In some examples, the free fatty acid is saturated. In some examples, the free fatty acid is unsaturated. In some embodiments, the free fatty acids are not derived from or produced by a mammal. Additional examples of free fatty acids are known in the art.

The compositions described herein, and the methods described herein can include the use of one or more phospholipids. Non-limiting examples of phospholipids include lecithin phospholipids (e.g., soy lecithin phospholipids, sunflower lecithin phospholipids, cotton lecithin phospholipids, rapeseed lecithin phospholipids. rice bran lecithin phospholipids, and corn lecithin phospholipids). Additional aspects of phospholipids are known in the art.

In some embodiments, the composition or method described herein can include the use of one or more different flavor compounds. Non-limiting examples of flavor compounds include δ-decalactone, ethyl butyrate, 2-furyl methyl ketone, 2,3-pentanedione, γ-undecalactone, and δ-undecalactone. Additional examples of flavor compounds include artificial flavors, e.g., chocolate, coffee, strawberry, almond, hazelnut, vanilla, green tea, Irish cream, and coconut flavoring. Additional examples of flavor compounds are known in the art.

A variety of different color balancing agents are known in the art. For example, a color balancing agent can be a compound from obtained from a plant (e.g., a monocot or a dicot). In some examples, the color balancing agent is a synthetic compound. In some examples, the color balancing agent is not obtained from or produced by a mammal or a mammalian cell. Non-limiting examples of color balancing agents include β-carotene and annatto.

In some embodiments, the composition or method described herein can further use a sweetening agent, comprising: a saccharide (e.g., a monosaccharide, a disaccharide, or a polysaccharide) or an artificial sweetener. Non-limiting examples of sweetening agents that are saccharides include glucose, mannose, maltose, fructose, galactose, lactose, sucrose, monatin, and tagatose. Additional examples of saccharides that can be used as a sweetening agent in any of the compositions or methods described herein are known in the art.

Non-limiting examples of sweetening agents that are artificial sweeteners include stevia, aspartame, cyclamate, saccharin, sucralose, mogrosides, brazzein, curculin, erythritol, glycyrrhizin, inulin, isomalt, lacititol, mabinlin, malititol, mannitol, miraculin, monatin, monelin, osladin, pentadin, sorbitol, thaumatin, xylitol, acesulfame potassium, advantame, alitame, aspartame-acesulfame, sodium cyclamate, dulcin, glucin, neohesperidin dihyrdochalcone, neotame, and P-4000. Additional artificial sweeteners that can be used as sweetening agents in any of the compositions or methods described herein are known in the art.

### Methods of Use

In some embodiments, there is provided a method for producing or preparing an edible composition comprising casein and/or whey aggregates.

In some embodiments, the method comprises providing a composition comprising a chimeric polypeptide comprising: (i) a first polypeptide being a polypeptide selected from: alpha S1 casein, alpha S2 casein, beta casein, whey polypeptide, or a functional analog thereof; and (ii) a second polypeptide being a kappa casein macropeptide, wherein the first polypeptide and the second polypeptide are interspaced by a cleavage site of a proteolytic enzyme, and contacting the composition with an effective amount of a proteolytic enzyme, thereby, producing the edible composition comprising the casein aggregates.

In some embodiments, the method comprises contacting the polypeptide of the invention, the composition of the invention, or both, with an effective amount of a proteolytic enzyme.

In some embodiments, the method comprises providing the polypeptide of the invention, the composition of the invention, or both.

In some embodiments, the edible composition comprising the casein aggregates, comprises a cheese or a curd.

As used herein, the term “contacting” refers to one of: adding, mixing, blending, stirring, diluting, incubating, or any combination thereof.

In some embodiments, contacting is in pH within the range of 1.0-11.0. In some embodiments, contacting is in pH within the range comprising: 1.0-2.0, 1.0-3.0, 1.0-4.0, 1.0-5.0, 1.0-6.0, 1.0-7.0, 1.0-8.0, 1.0-9.0, 1.0-10.0-1.0-11.0, 2.0-3.0, 2.0-4.0, 2.0-5.0, 2.0-6.0, 2.0-7.0, 2.0-8.0, 2.0-9.0, 2.0-10.0, 2.0-11.0, 3.0-4.0, 3.0-5.0, 3.0-6.0, 3.0-7.0, 3.0-8.0, 3.0-9.0, 3.0-10.0, 3.0-11.0, 4.0-5.0, 4.0-6.0, 4.0-7.0, 4.0-8.0, 4.0-9.0, 4.0-10.0, 4.0-11.0, 5.0-6.0, 5.0-7.0, 5.0-8.0, 5.0-9.0, 5.0-10.0, 5.0-11.0, 6.0-7.0, 6.0-8.0, 6.0-9.0, 6.0-10.0, 6.0-11.0, 7.0-8.0, 7.0-9.0, 7.0-10.0, 7.0-11.0, 8.0-9.0, 8.0-10.0, 8.0-11.0, 9.0-10.0, 9.0-11.0, or 10.0-11.0. Each possibility represent a separate embodiment of the invention.

In some embodiments, contacting is within a temperature range of 10-90° C. In some embodiments, contacting is in pH within the range comprising: 10-20° C., 10-30° C., 10-40° C., 10-50° C., 10-60° C., 10-70° C., 10-80° C., 10-90° C., 20-30° C., 20-40° C., 20-50° C., 20-60° C., 20-70° C., 20-80° C., 20-90° C., 30-40° C., 30-50° C., 30-60° C., 30-70° C., 30-80° C., 30-90° C., 40-50° C., 40-60° C., 40-70° C., 40-80° C., 40-90° C., 50-60° C., 50-70° C., 50-80° C., 50-90° C., 60-70° C., 60-80° C., 60-90° C., 70-80° C., 70-90° C., or 80-90° C. Each possibility represent a separate embodiment of the invention.

In some embodiments, contacting is under pH and temperature as disclosed herein.

In some embodiments, the method of the invention further comprises a step proceeding the contacting, comprising isolating the casein aggregates.

The term “isolating” comprises at least one of: curding, filtering, centrifugating, concentrating, precipitating, coagulating, or any combination thereof.

In some embodiments, coagulation of the polypeptide of the invention comprises a two-step process comprising: (a) a proteolytic stage comprising enzymatically cleaving the chimeric polypeptide to: alpha S1-, alpha S2-, beta-insoluble, and macropeptide-soluble in trichloroacetic acid (TCA); and (b) a coagulation stage, comprising precipitating at least one of: alpha S1, alpha S2 and beta, resulting in curd formation.

In some embodiments, a coagulation stage may require the addition of calcium or calcium ions, thereby enabling the precipitation of at least one of: alpha S1, alpha S2 and beta casein. In some embodiments, the precipitated alpha S1, alpha S2 or beta casein, precipitates as a calcium-bound casein.

### General

Any concentration ranges, percentage range, or ratio range recited herein are to be understood to include concentrations, percentages, or ratios of any integer within that range and fractions thereof, such as one tenth and one hundredth of an integer, unless otherwise indicated.

Any number range recited herein relating to any physical feature, such as polynucleotides and polypeptides, size, weight, or length, are to be understood to include any integer within the recited range, unless otherwise indicated.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an”, and “at least one”, are used interchangeably in this application.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

All publications, patents and patent applications mentioned in this specification are herein incorporated in their entirety by references into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### EXAMPLES

Generally, the nomenclature used herein and the laboratory procedures utilized in the present invention include immunological, chemical, molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Example 1

The inventors constructed a chimera casein protein that comprises a full length of one of: αS casein (αS1 or αS2) proteins, β casein (beta CSN), β-lactoglobulin (BLG), and α-lactalbumin (ALA), fused to the tail (n terminus) of κ casein, that is cleavable by the protease rennet. The construct is designed in such a way that no extra amino acids besides the: αS1 casein, αS2 casein, β casein, β-lactoglobulin or α-lactalbumin remain within the product after cleaving the κ tail (the κ macropeptide), i.e., the same amino acid (aa) sequence as the native protein. The chimeric protein is expressed in either mammalian cells or the yeast Pichia pastoris, organized in micelle, and secreted. The second goal is to demonstrate that the expressed chimeric protein is aggregated such that a matrix is formed (curd like) by treatment of rennet cleaving off the K tail and by reduction of pH.

Amino acids sequences of full-length wild type alpha S1 casein, alpha S2 casein, beta casein, and whey polypeptides:

A. CASA1_BOVIN Alpha-S1-casein (αS1), P02662:
(SEQ ID NO: 1)
MKLLILTCLVAVALARPKHPIKHQGLPQEVLNENLLRFFVAPFPE
VFGKEKVNELSKDIGSESTEDQAMEDIKQMEAESISSSEEIVPNS
VEQKHIQKEDVPSERYLGYLEQLLRLKKYKVPQLEIVPNSAEERL
HSMKEGIHAQQKEPMIGVNQELAYFYPELFRQFYQLDAYPSGAWY
YVPLGTQYTDAPSFSDIPNPIGSENSEKTTMPLW;
B. CASA2_BOVIN Alpha-S2-casein (αS1), P02663:
(SEQ ID NO: 2)
MKFFIFTCLLAVALAKNTMEHVSSSEESIISQETYKQEKNMAINP
SKENLCSTFCKEVVRNANEEEYSIGSSSEESAEVATEEVKITVDD
KHYQKALNEINQFYQKFPQYLQYLYQGPIVLNPWDQVKRNAVPIT
PTLNREQLSTSEENSKKTVDMESTEVFTKKTKLTEEEKNRLNFLK
KISQRYQKFALPQYLKTVYQHQKAMKPWIQPKTKVIPYVRYL;
C. Beta-casein (β casein or BCN)
CASB_BOVIN P02666:
(SEQ ID NO: 3)
MKVLILACLVALALARELEELNVPGEIVESLSSSEESITRINKKI
EKFQSEEQQQTEDELQDKIHPFAQTQSLVYPFPGPIPNSLPQNIP
PLTQTPVVVPPFLQPEVMGVSKVKEAMAPKHKEMPFPKYPVEPFT
ESQSLTLTDVENLHLPLPLLQSWMHQPHQPLPPTVMFPPQSVLSL
SQSKVLPVPQKAVPYPQRDMPIQAFLLYQEPVLGPVRGPFPIIV;
D. Beta Lactoglobulin (β lactoglobulin)
BLG BOVIN P02754:
(SEQ ID NO: 4)
MKCLLLALALTCGAQALIVTQTMKGLDIQKVAGTWYSLAMAASDI
SLLDAQSAPLRVYVEELKPTPEGDLEILLQKWENGECAQKKIIAE
KTKIPAVFKIDALNENKVLVLDTDYKKYLLFCMENSAEPEQSLAC
QCLVRTPEVDDEALEKFDKALKALPMHIRLSFNPTQLEEQCHI;
E. BLG with a signal peptide from Aspergillus
ORYZAE:
(SEQ ID NO: 5)
MKFFAIAALFAAAAVAQPNVISKRLIVTQTMKGLDIQKVAGTWYS
LAMAASDISLLDAQSAPLRVYVEELKPTPEGDLEILLQKWENGEC
AQKKIIAEKTKIPAVFKIDALNENKVLVLDTDYKKYLLFCMENSA
EPEQSLACQCLVRTPEVDDEALEKFDKALKALPMHIRLSFNPTQL
EEQCHI;
F. ALA Alpha-lactalbumin (α lactalbumin) P00711:
(SEQ ID NO: 6)
MMSFVSLLLVGILFHATQAEQLTKCEVFRELKDLKGYGGVSLPEW
VCTTFHTSGYDTQAIVQNNDSTEYGLFQINNKIWCKDDQNPHSSN
ICNISCDKFLDDDLTDDIMCVKKILDKVGINYWLAHKALCSEKLD
QWLCEKL;

Amino acids sequence of alpha S1 casein, alpha S2 casein, beta casein, and whey polypeptides, devoid of the amino acids located 3′ downstream to the last Phe (F) at the 3′ terminal end:

A. CASA1_BOVIN Alpha-S1-casein (αS1), truncated:
(SEQ ID NO: 7)
MKLLILTCLVAVALARPKHPIKHQGLPQEVLNENLLRFFVAPFPE
VFGKEKVNELSKDIGSESTEDQAMEDIKQMEAESISSSEEIVPNS
VEQKHIQKEDVPSERYLGYLEQLLRLKKYKVPQLEIVPNSAEERL
HSMKEGIHAQQKEPMIGVNQELAYFYPELFRQFYQLDAYPSGAWY
YVPLGTQYTDAPSF;
B. CASA2_BOVIN Alpha-S2-casein (αS1), truncated:
(SEQ ID NO: 8)
MKFFIFTCLLAVALAKNTMEHVSSSEESIISQETYKQEKNMAINP
SKENLCSTFCKEVVRNANEEEYSIGSSSEESAEVATEEVKITVDD
KHYQKALNEINQFYQKFPQYLQYLYQGPIVLNPWDQVKRNAVPIT
PTLNREQLSTSEENSKKTVDMESTEVFTKKTKLTEEEKNRLNFLK
KISQRYQKF;
C. Beta-casein (β casein or BCN)
CASB_BOVIN truncated:
(SEQ ID NO: 9)
MKVLILACLVALALARELEELNVPGEIVESLSSSEESITRINKKI
EKFQSEEQQQTEDELQDKIHPFAQTQSLVYPFPGPIPNSLPQNIP
PLTQTPVVVPPFLQPEVMGVSKVKEAMAPKHKEMPFPKYPVEPFT
ESQSLTLTDVENLHLPLPLLQSWMHQPHQPLPPTVMFPPQSVLSL
SQSKVLPVPQKAVPYPQRDMPIQAFLLYQEPVLGPVRGPF;
D. BLG BOVIN (β lactoglobulin) truncated:
(SEQ ID NO: 10)
MKCLLLALALTCGAQALIVTQTMKGLDIQKVAGTWYSLAMAASDI
SLLDAQSAPLRVYVEELKPTPEGDLEILLQKWENGECAQKKIIAE
KTKIPAVFKIDALNENKVLVLDTDYKKYLLFCMENSAEPEQSLAC
QCLVRTPEVDDEALEKFDKALKALPMHIRLSF;
E. BLG from A. ORYZAE_truncated:
(SEQ ID NO: 11)
MKFFAIAALFAAAAVAQPNVISKRLIVTQTMKGLDIQKVAGTWYS
LAMAASDISLLDAQSAPLRVYVEELKPTPEGDLEILLQKWENGEC
AQKKIIAEKTKIPAVFKIDALNENKVLVLDTDYKKYLLFCMENSA
EPEQSLACQCLVRTPEVDDEALEKFDKALKALPMHIRLSF;
F. ALA Alpha-lactalbumin (α lactalbumin) truncated:
(SEQ ID NO: 12)
MMSFVSLLLVGILFHATQAEQLTKCEVFRELKDLKGYGGVSLPEW
VCTTFHTSGYDTQAIVQNNDSTEYGLFQINNKIWCKDDQNPHSSN
ICNISCDKF;

κ casein sequences:

CASK_BOVIN Kappa-casein, P02668 (full length):
(SEQ ID NO: 13)
MMKSFFLVVTILALTLPFLGAQEQNQEQPIRCEKDERFFSDKIAK
YIPIQYVLSRYPSYGLNYYQQKPVALINNQFLPYPYYAKPAAVRS
PAQILQWQVLSNTVPAKSCQAQPTTMARHPHPHLSFMAIPPKKNQ
DKTEIPTINTIASGEPTSTPTTEAVESTVATLEDSPEVIESPPEI
NTVQVTSTAV;
Kappa casein macropeptide:
(SEQ ID NO: 14)
MAIPPKKNQDKTEIPTINTIASGEPTSTPTTEAVESTVATLEDSP
EVIESPPEINTVQVTSTAV;
Kappa casein macropeptide devoid of a cleavage site
(devoid of first methionine):
(SEQ ID NO: 15)
AIPPKKNQDKTEIPTINTIASGEPTSTPTTEAVESTVATLEDSPE
VIESPPEINTVQVTSTAV;

The suggested chimeric polypeptides include:

A. Polypeptide αS1 + KCN:
(SEQ ID NO: 16)
MKLLILTCLVAVALARPKHPIKHQGLPQEVLNENLLRFFVAPFPE
VFGKEKVNELSKDIGSESTEDQAMEDIKQMEAESISSSEEIVPNS
VEQKHIQKEDVPSERYLGYLEQLLRLKKYKVPQLEIVPNSAEERL
HSMKEGIHAQQKEPMIGVNQELAYFYPELFRQFYQLDAYPSGAWY
YVPLGTQYTDAPSFMAIPPKKNQDKTEIPTINTIASGEPTSTPTT
EAVESTVATLEDSPEVIESPPEINTVQVTSTAV;
B. Polypeptide αS2 + KCN:
(SEQ ID NO: 17)
MKFFIFTCLLAVALAKNTMEHVSSSEESIISQETYKQEKNMAINP
SKENLCSTFCKEVVRNANEEEYSIGSSSEESAEVATEEVKITVDD
KHYQKALNEINQFYQKFPQYLQYLYQGPIVLNPWDQVKRNAVPIT
PTLNREQLSTSEENSKKTVDMESTEVFTKKTKLTEEEKNRLNFLK
KISQRYQKFMAIPPKKNQDKTEIPTINTIASGEPTSTPTTEAVES
TVATLEDSPEVIESPPEINTVQVTSTAV;
C. Polypeptide BCN (β-casein) + KCN:
(SEQ ID NO: 18)
MKVLILACLVALALARELEELNVPGEIVESLSSSEESITRINKKI
EKFQSEEQQQTEDELQDKIHPFAQTQSLVYPFPGPIPNSLPQNIP
PLTQTPVVVPPFLQPEVMGVSKVKEAMAPKHKEMPFPKYPVEPFT
ESQSLTLTDVENLHLPLPLLQSWMHQPHQPLPPTVMFPPQSVLSL
SQSKVLPVPQKAVPYPQRDMPIQAFLLYQEPVLGPVRGPFMAIPP
KKNQDKTEIPTINTIASGEPTSTPTTEAVESTVATLEDSPEVIES
PPEINTVQVTSTAV;
D. Polypeptide BLG (BOVIN) + KCN:
(SEQ ID NO: 19)
MKCLLLALALTCGAQALIVTQTMKGLDIQKVAGTWYSLAMAASDI
SLLDAQSAPLRVYVEELKPTPEGDLEILLQKWENGECAQKKIIAE
KTKIPAVFKIDALNENKVLVLDTDYKKYLLFCMENSAEPEQSLAC
QCLVRTPEVDDEALEKFDKALKALPMHIRLSFMAIPPKKNQDKTE
IPTINTIASGEPTSTPTTEAVESTVATLEDSPEVIESPPEINTVQ
VTSTAV;
E. Polypeptide IMG1 BLG (A. ORYZAE) + KCN:
(SEQ ID NO: 20)
MKFFAIAALFAAAAVAQPNVISKRLIVTQTMKGLDIQKVAGTWYS
LAMAASDISLLDAQSAPLRVYVEELKPTPEGDLEILLQKWENGEC
AQKKIIAEKTKIPAVFKIDALNENKVLVLDTDYKKYLLFCMENSA
EPEQSLACQCLVRTPEVDDEALEKFDKALKALPMHIRLSFMAIPP
KKNQDKTEIPTINTIASGEPTSTPTTEAVESTVATLEDSPEVIES
PPEINTVQVTSTAV;
F. Polypeptide ALA (α-lactalbumin) + KCN:
(SEQ ID NO: 21)
MMSFVSLLLVGILFHATQAEQLTKCEVFRELKDLKGYGGVSLPEW
VCTTFHTSGYDTQAIVQNNDSTEYGLFQINNKIWCKDDQNPHSSN
ICNISCDKFMAIPPKKNQDKTEIPTINTIASGEPTSTPTTEAVES
TVATLEDSPEVIESPPEINTVQVTSTAV;

Nucleic acid sequences of full-length wild type alpha S1 casein, alpha S2 casein, beta casein, and a whey polypeptide:

A. cDNA of CASA1_BOVIN Alpha-S1-casein (αS1),
ENA|CAB57792|CAB57792.1 Bos taurus
(cattle):
(SEQ ID NO: 22)
ATGAAACTTCTCATCCTTACCTGTCTTGTGGCTGTTGCTCTTGCT
AGGCCTAAACATCCTATCAAGCACCAAGGACTCCCTCAAGAAGTC
CTCAATGAAAATTTACTCAGGTTTTTTGTGGCACCTTTTCCAGAA
GTGTTTGGAAAGGAGAAGGTCAATGAACTGAGCAAGGATATTGGG
AGTGAATCAACTGAGGATCAAGCCATGGAAGATATTAAGCAAATG
GAAGCTGAAAGCATTTCGTCAAGTGAGGAAATTGTTCCCAATAGT
GTTGAGCAGAAGCACATTCAAAAGGAAGATGTGCCCTCTGAGCGT
TACCTGGGTTATCTGGAACAGCTTCTCAGACTGAAAAAATACAAA
GTACCCCAGCTGGAAATTGTTCCCAATAGTGCTGAGGAACGACTT
CACAGTATGAAAGAGGGAATCCATGCCCAACAGAAAGAACCTATG
ATAGGAGTGAATCAGGAACTGGCCTACTTCTACCCTGAGCTTTTC
AGACAATTCTACCAGCTGGATGCCTATCCATCTGGTGCCTGGTAT
TACGTTCCACTAGGCACACAATACACTGATGCCCCATCATTCTCT
GACATCCCTAATCCTATTGGCTCTGAGAACAGTGAAAAGACTACT
ATGCCACTGTGGTGA;
B. cDNA of CASA2_BOVIN Alpha-S2-casein (αS2),
ENA|AAA30479|AAA30479.1 Bos taurus
(cattle)
(SEQ ID NO: 23)
ATGAAGTTCTTCATCTTTACCTGCCTTTTGGCTGTTGCCCTTGCA
AAGAATACGATGGAACATGTCTCCTCCAGTGAGGAATCTATCATC
TCCCAGGAAACATATAAGCAGGAAAAGAATATGGCCATTAATCCC
AGCAAGGAGAACCTTTGCTCCACATTCTGCAAGGAAGTTGTAAGG
AACGCAAATGAAGAGGAATATTCTATCGGCTCATCTAGTGAGGAA
TCTGCTGAAGTTGCCACTGAGGAAGTTAAGATTACTGTGGACGAT
AAGCACTACCAGAAAGCACTGAATGAAATCAATCAGTTTTATCAG
AAGTTCCCCCAGTATCTCCAGTATCTGTATCAAGGTCCAATTGTT
TTGAACCCATGGGATCAGGTTAAGAGGAATGCTGTTCCCATTACT
CCCACTCTGAACAGAGAGCAGCTCTCCACCAGTGAGGAAAATTCA
AAGAAGACCGTTGACATGGAATCAACAGAAGTATTCACTAAGAAA
ACTAAACTGACTGAAGAAGAAAAGAATCGCCTAAATTTTCTGAAA
AAAATCAGCCAGCGTTACCAGAAATTCGCCTTGCCCCAGTATCTC
AAGACTGTTTATCAGCATCAGAAAGCTATGAAGCCATGGATTCAA
CCTAAGACAAAGGTTATTCCCTATGTGAGGTACCTTTAA;
C. cDNA of Beta-casein (β casein or BCN)
CASB_BOVIN, ENAJAAA30480:
(SEQ ID NO: 24)
ATGAAGGTCCTCATCCTTGCCTGCCTGGTGGCTCTGGCCCTTGCA
AGAGAGCTGGAAGAACTCAATGTACCTGGTGAGATTGTGGAAAGC
CTTTCAAGCAGCGAGGAATCAATTACACGCATCAATAAGAAAATT
GAGAAGTTTCAGAGTGAGGAACAGCAGCAAACAGAGGATGAACTC
CAGGATAAAATCCACCCCTTTGCCCAGACACAGTCTCTAGTCTAT
CCCTTCCCTGGGCCCATCCCTAACAGCCTCCCACAAAACATCCCT
CCTCTTACTCAAACCCCTGTGGTGGTGCCGCCTTTCCTTCAGCCT
GAAGTAATGGGAGTCTCCAAAGTGAAGGAGGCTATGGCCCCTAAG
CACAAAGAAATGCCCTTCCCTAAATATCCAGTTGAGCCCTTTACT
GAAAGCCAGAGCCTGACTCTCACTGATGTTGAAAATCTGCACCTT
CCTCTGCCTCTGCTCCAGTCTTGGATGCACCAGCCTCACCAGCCT
CTTCCTCCAACTGTCATGTTTCCTCCTCAGTCCGTGCTGTCCCTT
TCTCAGTCCAAAGTCCTGCCTGTTCCCCAGAAAGCAGTGCCCTAT
CCCCAGAGAGATATGCCCATTCAGGCCTTTCTGCTGTACCAGGAG
CCTGTACTCGGTCCTGTCCGGGGACCCTTCCCTATTATTGTCTAA;
D. cDNA of beta lactoglobulin ENAJAAI08214|
AAI08214.1 Bos taurus (cattle):
(SEQ ID NO: 25)
ATGAAGTGCCTCCTGCTTGCCCTGGCCCTCACTTGTGGCGCCCAG
GCCCTCATTGTCACCCAGACCATGAAGGGCCTGGATATCCAGAAG
GTGGCGGGGACTTGGTACTCCTTGGCCATGGCGGCCAGCGACATC
TCCCTGCTGGACGCCCAGAGTGCCCCCCTGAGAGTGTATGTGGAG
GAGCTGAAGCCCACCCCTGAGGGCGACCTGGAGATCCTGCTGCAG
AAATGGGAGAACGGTGAGTGTGCTCAGAAGAAGATCATTGCAGAA
AAAACCAAGATCCCTGCGGTGTTCAAGATCGATGCCTTGAATGAG
AACAAAGTCCTTGTGCTGGACACCGACTACAAAAAGTACCTGCTC
TTCTGCATGGAGAACAGTGCTGAGCCCGAGCAAAGCCTGGCCTGC
CAGTGCCTGGTCAGGACCCCGGAGGTGGACGACGAGGCCCTGGAG
AAATTCGACAAAGCCCTCAAGGCCCTGCCCATGCACATCCGGCTG
TCCTTCAACCCAACCCAGCTGGAGGAGCAGTGCCACATC;
E. cDNA of BLG_A. ORYZAE_IMG1:
(SEQ ID NO: 26)
ATGAAGTTTTTCGCCATTGCCGCCCTATTTGCCGCCGCTGCCGTT
GCCCAGCCTAACGTCATCTCGAAGCGGCTCATCGTCACCCAGACC
ATGAAGGGCCTCGACATCCAGAAGGTCGCCGGCACCTGGTACAGC
CTCGCCATGGCCGCCAGCGACATCAGCCTGCTCGACGCCCAGAGC
GCCCCTCTCCGCGTCTACGTCGAGGAGCTGAAGCCCACGCCTGAG
GGCGACCTCGAGATCCTCCTGCAGAAGTGGGAGAACGGCGAGTGC
GCCCAGAAGAAGATCATTGCCGAGAAGACGAAGATCCCCGCCGTG
TTCAAGATCGACGCCCTCAACGAGAACAAGGTCCTCGTCCTCGAC
ACCGACTACAAGAAGTACCTCCTGTTCTGCATGGAGAACTCCGCC
GAGCCTGAGCAGAGCCTCGCCTGCCAGTGCCTCGTTCGCACGCCC
GAGGTCGACGACGAGGCCCTCGAGAAGTTCGACAAGGCCCTCAAG
GCTCTCCCCATGCACATCCGCCTCAGCTTCAACCCCACGCAGCTC
GAGGAGCAGTGCCACATC;
F. cDNA of ENAJA CI62509|A CI62509.1
Bos taurus (cattle) ALA (α lactalbumin):
(SEQ ID NO: 27)
ATGATGTCCTTTGTCTCTCTGCTCCTGGTAGGCATCCTATTCCAT
GCCACCCAGGCTGAACAGTTAACAAAATGTGAGGTGTTCCGGGAG
CTGAAAGACTTGAAGGGCTACGGAGGTGTCAGTTTGCCTGAATGG
GTCTGTACCACGTTTCATACCAGTGGTTATGACACACAAGCCATA
GTACAAAACAATGACAGCACAGAATATGGACTCTTCCAGATAAAT
AATAAAATTTGGTGCAAAGACGACCAGAACCCTCACTCAAGCAAC
ATCTGTAACATCTCCTGTGACAAGTTCCTGGATGATGATCTTACT
GATGACATTATGTGTGTCAAGAAGATTCTGGATAAAGTAGGAATT
AACTACTGGTTGGCCCATAAAGCACTCTGTTCTGAGAAGCTGGAT
CAGTGGCTCTGTGAGAAGTTGTGA;

Nucleic acids sequence of alpha S1 casein, alpha S2 casein, beta casein, and a whey polypeptide, encoding to a truncated polypeptide devoid of the amino acids located 3′ downstream to the last Phe (F) at the 3′ terminal end:

A. CASA1_BOVIN Alpha-S1-casein (αS1), truncated:
(SEQ ID NO: 28)
ATGAAACTTCTCATCCTTACCTGTCTTGTGGCTGTTGCTCTTGCT
AGGCCTAAACATCCTATCAAGCACCAAGGACTCCCTCAAGAAGTC
CTCAATGAAAATTTACTCAGGTTTTTTGTGGCACCTTTTCCAGAA
GTGTTTGGAAAGGAGAAGGTCAATGAACTGAGCAAGGATATTGGG
AGTGAATCAACTGAGGATCAAGCCATGGAAGATATTAAGCAAATG
GAAGCTGAAAGCATTTCGTCAAGTGAGGAAATTGTTCCCAATAGT
GTTGAGCAGAAGCACATTCAAAAGGAAGATGTGCCCTCTGAGCGT
TACCTGGGTTATCTGGAACAGCTTCTCAGACTGAAAAAATACAAA
GTACCCCAGCTGGAAATTGTTCCCAATAGTGCTGAGGAACGACTT
CACAGTATGAAAGAGGGAATCCATGCCCAACAGAAAGAACCTATG
ATAGGAGTGAATCAGGAACTGGCCTACTTCTACCCTGAGCTTTTC
AGACAATTCTACCAGCTGGATGCCTATCCATCTGGTGCCTGGTAT
TACGTTCCACTAGGCACACAATACACTGATGCCCCATCATTC;
B. CASA2_BOVIN Alpha-S2-casein (αS2), truncated:
(SEQ ID NO: 29)
ATGAAGTTCTTCATCTTTACCTGCCTTTTGGCTGTTGCCCTTGCA
AAGAATACGATGGAACATGTCTCCTCCAGTGAGGAATCTATCATC
TCCCAGGAAACATATAAGCAGGAAAAGAATATGGCCATTAATCCC
AGCAAGGAGAACCTTTGCTCCACATTCTGCAAGGAAGTTGTAAGG
AACGCAAATGAAGAGGAATATTCTATCGGCTCATCTAGTGAGGAA
TCTGCTGAAGTTGCCACTGAGGAAGTTAAGATTACTGTGGACGAT
AAGCACTACCAGAAAGCACTGAATGAAATCAATCAGTTTTATCAG
AAGTTCCCCCAGTATCTCCAGTATCTGTATCAAGGTCCAATTGTT
TTGAACCCATGGGATCAGGTTAAGAGGAATGCTGTTCCCATTACT
CCCACTCTGAACAGAGAGCAGCTCTCCACCAGTGAGGAAAATTCA
AAGAAGACCGTTGACATGGAATCAACAGAAGTATTCACTAAGAAA
ACTAAACTGACTGAAGAAGAAAAGAATCGCCTAAATTTTCTGAAA
AAAATCAGCCAGCGTTACCAGAAATTC;
C. Beta-casein ( casein or BCN) CASB_BOVIN,
truncated:
(SEQ ID NO: 30)
ATGAAGGTCCTCATCCTTGCCTGCCTGGTGGCTCTGGCCCTTGCA
AGAGAGCTGGAAGAACTCAATGTACCTGGTGAGATTGTGGAAAGC
CTTTCAAGCAGCGAGGAATCAATTACACGCATCAATAAGAAAATT
GAGAAGTTTCAGAGTGAGGAACAGCAGCAAACAGAGGATGAACTC
CAGGATAAAATCCACCCCTTTGCCCAGACACAGTCTCTAGTCTAT
CCCTTCCCTGGGCCCATCCCTAACAGCCTCCCACAAAACATCCCT
CCTCTTACTCAAACCCCTGTGGTGGTGCCGCCTTTCCTTCAGCCT
GAAGTAATGGGAGTCTCCAAAGTGAAGGAGGCTATGGCCCCTAAG
CACAAAGAAATGCCCTTCCCTAAATATCCAGTTGAGCCCTTTACT
GAAAGCCAGAGCCTGACTCTCACTGATGTTGAAAATCTGCACCTT
CCTCTGCCTCTGCTCCAGTCTTGGATGCACCAGCCTCACCAGCCT
CTTCCTCCAACTGTCATGTTTCCTCCTCAGTCCGTGCTGTCCCTT
TCTCAGTCCAAAGTCCTGCCTGTTCCCCAGAAAGCAGTGCCCTAT
CCCCAGAGAGATATGCCCATTCAGGCCTTTCTGCTGTACCAGGAG
CCTGTACTCGGTCCTGTCCGGGGACCCTTC;
D. BLG BOVIN (β lactoglobulin), truncated:
(SEQ ID NO: 31)
ATGAAGTGCCTCCTGCTTGCCCTGGCCCTCACTTGTGGCGCCCAG
GCCCTCATTGTCACCCAGACCATGAAGGGCCTGGATATCCAGAAG
GTGGCGGGGACTTGGTACTCCTTGGCCATGGCGGCCAGCGACATC
TCCCTGCTGGACGCCCAGAGTGCCCCCCTGAGAGTGTATGTGGAG
GAGCTGAAGCCCACCCCTGAGGGCGACCTGGAGATCCTGCTGCAG
AAATGGGAGAACGGTGAGTGTGCTCAGAAGAAGATCATTGCAGAA
AAAACCAAGATCCCTGCGGTGTTCAAGATCGATGCCTTGAATGAG
AACAAAGTCCTTGTGCTGGACACCGACTACAAAAAGTACCTGCTC
TTCTGCATGGAGAACAGTGCTGAGCCCGAGCAAAGCCTGGCCTGC
CAGTGCCTGGTCAGGACCCCGGAGGTGGACGACGAGGCCCTGGAG
AAATTCGACAAAGCCCTCAAGGCCCTGCCCATGCACATCCGGCTG
TCCTTC;
E. BLG from A. ORYZAE,_truncated:
(SEQ ID NO: 32)
ATGAAGTTTTTCGCCATTGCCGCCCTATTTGCCGCCGCTGCCGTT
GCCCAGCCTAACGTCATCTCGAAGCGGCTCATCGTCACCCAGACC
ATGAAGGGCCTCGACATCCAGAAGGTCGCCGGCACCTGGTACAGC
CTCGCCATGGCCGCCAGCGACATCAGCCTGCTCGACGCCCAGAGC
GCCCCTCTCCGCGTCTACGTCGAGGAGCTGAAGCCCACGCCTGAG
GGCGACCTCGAGATCCTCCTGCAGAAGTGGGAGAACGGCGAGTGC
GCCCAGAAGAAGATCATTGCCGAGAAGACGAAGATCCCCGCCGTG
TTCAAGATCGACGCCCTCAACGAGAACAAGGTCCTCGTCCTCGAC
ACCGACTACAAGAAGTACCTCCTGTTCTGCATGGAGAACTCCGCC
GAGCCTGAGCAGAGCCTCGCCTGCCAGTGCCTCGTTCGCACGCCC
GAGGTCGACGACGAGGCCCTCGAGAAGTTCGACAAGGCCCTCAAG
GCTCTCCCCATGCACATCCGCCTCAGCTTC;
F. ALA Alpha-lactalbumin (α lactalbumin),
truncated:
(SEQ ID NO: 33)
ATGATGTCCTTTGTCTCTCTGCTCCTGGTAGGCATCCTATTCCAT
GCCACCCAGGCTGAACAGTTAACAAAATGTGAGGTGTTCCGGGAG
CTGAAAGACTTGAAGGGCTACGGAGGTGTCAGTTTGCCTGAATGG
GTCTGTACCACGTTTCATACCAGTGGTTATGACACACAAGCCATA
GTACAAAACAATGACAGCACAGAATATGGACTCTTCCAGATAAAT
AATAAAATTTGGTGCAAAGACGACCAGAACCCTCACTCAAGCAAC
ATCTGTAACATCTCCTGTGACAAGTTC;
casein nucleic acid sequences:
CASK_BOVIN Kappa-casein, (full length)
ENA/AAQ87922|AAQ87922.1 Bos taurus (cattle):
(SEQ ID NO: 34)
ATGATGAAGAGTTTTTTCCTAGTTGTGACTATCCTGGCATTAACC
CTGCCATTTTTGGGTGCCCAGGAGCAAAACCAAGAACAACCAATA
CGCTGTGAGAAAGATGAAAGATTCTTCAGTGACAAAATAGCCAAA
TATATCCCAATTCAGTATGTGCTGAGTAGGTATCCTAGTTATGGA
CTCAATTACTACCAACAGAAACCAGTTGCACTAATTAATAATCAA
TTTCTGCCATACCCATATTATGCAAAGCCAGCTGCAGTTAGGTCA
CCTGCCCAAATTCTTCAATGGCAAGTTTTGTCAAATACTGTGCCT
GCCAAGTCCTGCCAAGCCCAGCCAACTACCATGGCACGTCACCCA
CACCCACATTTATCATTTATGGCCATTCCACCAAAGAAAAATCAG
GATAAAACAGAAATCCCTACCATCAATACCATTGCTAGTGGTGAG
CCTACAAGTACACCTACCACCGAAGCAGTAGAGAGCACTGTAGCT
ACTCTAGAAGATTCTCCAGAAGTTATTGAGAGCCCACCTGAGATC
AACACAGTCCAAGTTACTTCAACTGCAGTCTAA;
Kappa casein macropeptide cDNA AAQ87922:
(SEQ ID NO: 35)
ATGGCCATTCCACCAAAGAAAAATCAGGATAAAACAGAAATCCCT
ACCATCAATACCATTGCTAGTGGTGAGCCTACAAGTACACCTACC
ACCGAAGCAGTAGAGAGCACTGTAGCTACTCTAGAAGATTCTCCA
GAAGTTATTGAGAGCCCACCTGAGATCAACACAGTCCAAGTTACT
TCAACTGCAGTC;
Kappa casein macropeptide devoid of a
cleavage site:
(SEQ ID NO: 36)
ATGGCCATTCCACCAAAGAAAAATCAGGATAAAACAGAAATCCCT
ACCATCAATACCATTGCTAGTGGTGAGCCTACAAGTACACCTACC
ACCGAAGCAGTAGAGAGCACTGTAGCTACTCTAGAAGATTCTCCA
GAAGTTATTGAGAGCCCACCTGAGATCAACACAGTCCAAGTTACT
TCAACTGCAGTC;

Nucleic acid sequences of the suggested chimeric polypeptides:

A. cDNA αS1 + KCN:
(SEQ ID NO: 37)
ATGAAACTTCTCATCCTTACCTGTCTTGTGGCTGTTGCTCTTGCT
AGGCCTAAACATCCTATCAAGCACCAAGGACTCCCTCAAGAAGTC
CTCAATGAAAATTTACTCAGGTTTTTTGTGGCACCTTTTCCAGAA
GTGTTTGGAAAGGAGAAGGTCAATGAACTGAGCAAGGATATTGGG
AGTGAATCAACTGAGGATCAAGCCATGGAAGATATTAAGCAAATG
GAAGCTGAAAGCATTTCGTCAAGTGAGGAAATTGTTCCCAATAGT
GTTGAGCAGAAGCACATTCAAAAGGAAGATGTGCCCTCTGAGCGT
TACCTGGGTTATCTGGAACAGCTTCTCAGACTGAAAAAATACAAA
GTACCCCAGCTGGAAATTGTTCCCAATAGTGCTGAGGAACGACTT
CACAGTATGAAAGAGGGAATCCATGCCCAACAGAAAGAACCTATG
ATAGGAGTGAATCAGGAACTGGCCTACTTCTACCCTGAGCTTTTC
AGACAATTCTACCAGCTGGATGCCTATCCATCTGGTGCCTGGTAT
TACGTTCCACTAGGCACACAATACACTGATGCCCCATCATTCATG
GCCATTCCACCAAAGAAAAATCAGGATAAAACAGAAATCCCTACC
ATCAATACCATTGCTAGTGGTGAGCCTACAAGTACACCTACCACC
GAAGCAGTAGAGAGCACTGTAGCTACTCTAGAAGATTCTCCAGAA
GTTATTGAGAGCCCACCTGAGATCAACACAGTCCAAGTTACTTCA
ACTGCAGTC;
B. cDNA αS2 + KCN:
(SEQ ID NO: 38)
ATGAAGTTCTTCATCTTTACCTGCCTTTTGGCTGTTGCCCTTGCA
AAGAATACGATGGAACATGTCTCCTCCAGTGAGGAATCTATCATC
TCCCAGGAAACATATAAGCAGGAAAAGAATATGGCCATTAATCCC
AGCAAGGAGAACCTTTGCTCCACATTCTGCAAGGAAGTTGTAAGG
AACGCAAATGAAGAGGAATATTCTATCGGCTCATCTAGTGAGGAA
TCTGCTGAAGTTGCCACTGAGGAAGTTAAGATTACTGTGGACGAT
AAGCACTACCAGAAAGCACTGAATGAAATCAATCAGTTTTATCAG
AAGTTCCCCCAGTATCTCCAGTATCTGTATCAAGGTCCAATTGTT
TTGAACCCATGGGATCAGGTTAAGAGGAATGCTGTTCCCATTACT
CCCACTCTGAACAGAGAGCAGCTCTCCACCAGTGAGGAAAATTCA
AAGAAGACCGTTGACATGGAATCAACAGAAGTATTCACTAAGAAA
ACTAAACTGACTGAAGAAGAAAAGAATCGCCTAAATTTTCTGAAA
AAAATCAGCCAGCGTTACCAGAAATTCATGGCCATTCCACCAAAG
AAAAATCAGGATAAAACAGAAATCCCTACCATCAATACCATTGCT
AGTGGTGAGCCTACAAGTACACCTACCACCGAAGCAGTAGAGAGC
ACTGTAGCTACTCTAGAAGATTCTCCAGAAGTTATTGAGAGCCCA
CCTGAGATCAACACAGTCCAAGTTACTTCAACTGCAGTC;
C. cDNA BCN (β-casein) + KCN:
(SEQ ID NO: 39)
ATGAAGGTCCTCATCCTTGCCTGCCTGGTGGCTCTGGCCCTTGCA
AGAGAGCTGGAAGAACTCAATGTACCTGGTGAGATTGTGGAAAGC
CTTTCAAGCAGCGAGGAATCAATTACACGCATCAATAAGAAAATT
GAGAAGTTTCAGAGTGAGGAACAGCAGCAAACAGAGGATGAACTC
CAGGATAAAATCCACCCCTTTGCCCAGACACAGTCTCTAGTCTAT
CCCTTCCCTGGGCCCATCCCTAACAGCCTCCCACAAAACATCCCT
CCTCTTACTCAAACCCCTGTGGTGGTGCCGCCTTTCCTTCAGCCT
GAAGTAATGGGAGTCTCCAAAGTGAAGGAGGCTATGGCCCCTAAG
CACAAAGAAATGCCCTTCCCTAAATATCCAGTTGAGCCCTTTACT
GAAAGCCAGAGCCTGACTCTCACTGATGTTGAAAATCTGCACCTT
CCTCTGCCTCTGCTCCAGTCTTGGATGCACCAGCCTCACCAGCCT
CTTCCTCCAACTGTCATGTTTCCTCCTCAGTCCGTGCTGTCCCTT
TCTCAGTCCAAAGTCCTGCCTGTTCCCCAGAAAGCAGTGCCCTAT
CCCCAGAGAGATATGCCCATTCAGGCCTTTCTGCTGTACCAGGAG
CCTGTACTCGGTCCTGTCCGGGGACCCTTCATGGCCATTCCACCA
AAGAAAAATCAGGATAAAACAGAAATCCCTACCATCAATACCATT
GCTAGTGGTGAGCCTACAAGTACACCTACCACCGAAGCAGTAGAG
AGCACTGTAGCTACTCTAGAAGATTCTCCAGAAGTTATTGAGAGC
CCACCTGAGATCAACACAGTCCAAGTTACTTCAACTGCAGTC;
D. cDNA BLG (BOVIN) + KCN:
(SEQ ID NO: 40)
ATGAAGTGCCTCCTGCTTGCCCTGGCCCTCACTTGTGGCGCCCAG
GCCCTCATTGTCACCCAGACCATGAAGGGCCTGGATATCCAGAAG
GTGGCGGGGACTTGGTACTCCTTGGCCATGGCGGCCAGCGACATC
TCCCTGCTGGACGCCCAGAGTGCCCCCCTGAGAGTGTATGTGGAG
GAGCTGAAGCCCACCCCTGAGGGCGACCTGGAGATCCTGCTGCAG
AAATGGGAGAACGGTGAGTGTGCTCAGAAGAAGATCATTGCAGAA
AAAACCAAGATCCCTGCGGTGTTCAAGATCGATGCCTTGAATGAG
AACAAAGTCCTTGTGCTGGACACCGACTACAAAAAGTACCTGCTC
TTCTGCATGGAGAACAGTGCTGAGCCCGAGCAAAGCCTGGCCTGC
CAGTGCCTGGTCAGGACCCCGGAGGTGGACGACGAGGCCCTGGAG
AAATTCGACAAAGCCCTCAAGGCCCTGCCCATGCACATCCGGCTG
TCCTTCATGGCCATTCCACCAAAGAAAAATCAGGATAAAACAGAA
ATCCCTACCATCAATACCATTGCTAGTGGTGAGCCTACAAGTACA
CCTACCACCGAAGCAGTAGAGAGCACTGTAGCTACTCTAGAAGAT
TCTCCAGAAGTTATTGAGAGCCCACCTGAGATCAACACAGTCCAA
GTTACTTCAACTGCAGTC;
E. cDNA IMG1 BLG (A. ORYZAE) + KCN:
(SEQ ID NO: 41)
ATGAAGTTTTTCGCCATTGCCGCCCTATTTGCCGCCGCTGCCGTT
GCCCAGCCTAACGTCATCTCGAAGCGGCTCATCGTCACCCAGACC
ATGAAGGGCCTCGACATCCAGAAGGTCGCCGGCACCTGGTACAGC
CTCGCCATGGCCGCCAGCGACATCAGCCTGCTCGACGCCCAGAGC
GCCCCTCTCCGCGTCTACGTCGAGGAGCTGAAGCCCACGCCTGAG
GGCGACCTCGAGATCCTCCTGCAGAAGTGGGAGAACGGCGAGTGC
GCCCAGAAGAAGATCATTGCCGAGAAGACGAAGATCCCCGCCGTG
TTCAAGATCGACGCCCTCAACGAGAACAAGGTCCTCGTCCTCGAC
ACCGACTACAAGAAGTACCTCCTGTTCTGCATGGAGAACTCCGCC
GAGCCTGAGCAGAGCCTCGCCTGCCAGTGCCTCGTTCGCACGCCC
GAGGTCGACGACGAGGCCCTCGAGAAGTTCGACAAGGCCCTCAAG
GCTCTCCCCATGCACATCCGCCTCAGCTTCATGGCCATCCCCCCC
AAGAAGAACCAGGACAAGACCGAGATCCCCACCATCAACACCATC
GCCTCCGGCGAGCCCACCTCCACCCCCACCACCGAGGCCGTCGAG
TCCACCGTCGCCACCCTGGAGGACTCCCCCGAGGTCATCGAGTCC
CCCCCCGAGATCAACACCGTCCAGGTCACCTCCACCGCCGTC;
F. cDNA ALA (α-lactalbumin) + KCN:
(SEQ ID NO: 42)
ATGATGTCCTTTGTCTCTCTGCTCCTGGTAGGCATCCTATTCCAT
GCCACCCAGGCTGAACAGTTAACAAAATGTGAGGTGTTCCGGGAG
CTGAAAGACTTGAAGGGCTACGGAGGTGTCAGTTTGCCTGAATGG
GTCTGTACCACGTTTCATACCAGTGGTTATGACACACAAGCCATA
GTACAAAACAATGACAGCACAGAATATGGACTCTTCCAGATAAAT
AATAAAATTTGGTGCAAAGACGACCAGAACCCTCACTCAAGCAAC
ATCTGTAACATCTCCTGTGACAAGTTCATGGCCATTCCACCAAAG
AAAAATCAGGATAAAACAGAAATCCCTACCATCAATACCATTGCT
AGTGGTGAGCCTACAAGTACACCTACCACCGAAGCAGTAGAGAGC
ACTGTAGCTACTCTAGAAGATTCTCCAGAAGTTATTGAGAGCCCA
CCTGAGATCAACACAGTCCAAGTTACTTCAACTGCAGTC.

### Materials and Methods

1) Design constructs: αS1casein-fusion κ, αS2casein-fusion κ, β-casein-fusion κ, β-lactoglobulin-fusion κ, and α-lactalbumin-fusion κ.
2) Expression test in CHO/293 cells or in Pichia;
3) Purification of 2-3 different proteins;
4) Test biochemical activity to determine micelle size;
5) Develop Biochemical test to assess aggregation to form a matrix when treat with rennet and lower pH; and
6) Select lead chimeric candidate and produce 0.5-1 g purified protein.

Further, wild type (WT) A. oryzae (Rib40) strain was transformed with synthetic expression cassette of a heterologous chimeric protein (Bos taurus originated) BLG:Kappa-Casein-Glycomacropeptide, in a specific designated chromosomal location using homologous recombination event. Positive single spore isolated colonies were confirmed by PCR and pyrG marker acquired resistance.

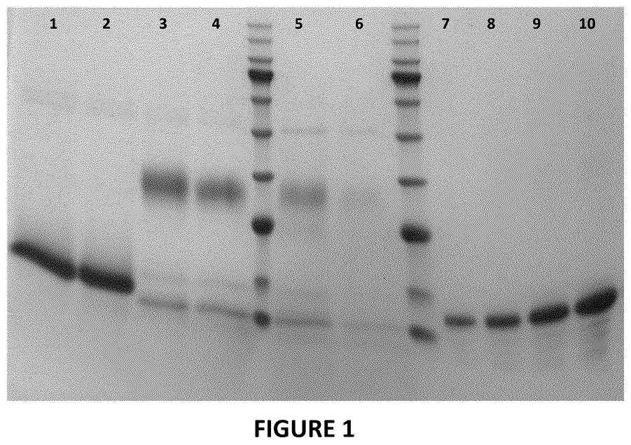

Chimeric protein expression evaluation: colonies spores [200 spores/ml] were inoculated in 250 ml BDMY (2% Glucose) media in in 1 L bottom baffled flasks at 28° C., 150 RPM. Samples were taken 72 hours post induction (hpi, 3 days) and analyzed in SDS-PSGE gel (compared to known BLG standards): 20 μl samples (unfiltered or after 0.2 μm filter) were resolved on 14% SDS-PAGE gels, and Coomasie stained (FIG. 1).

All publications, patents and patent applications mentioned in this specification are herein incorporated in their entirety by references into the specification, to the same extent as if each individual publication, patent, or patent application was specifically and individually indicated to be incorporated herein by reference. In addition, citation, or identification of any reference in this application shall not be construed as an admission that such reference is available as prior art to the present invention. To the extent that section headings are used, they should not be construed as necessarily limiting.

1. A chimeric polynucleotide comprising:
a) a first nucleic acid sequence encoding a first polypeptide selected from the group consisting of: alpha S1 casein, alpha S2 casein, beta casein, whey polypeptide, and any functional analog thereof;
b) a second nucleic acid sequence encoding a cleavage site of a proteolytic enzyme; and
c) a third nucleic acid sequence encoding a kappa casein macropeptide. wherein said second nucleic acid sequence is positioned between said first and third nucleic acid sequences, and wherein said first, second, and third nucleic acid sequences are operably linked.

2. The chimeric polynucleotide of claim 1, wherein said whey polypeptide is beta lactoglobulin or alpha lactalbumin.

3. The chimeric polynucleotide of claim 1, wherein said proteolytic enzyme belongs to the family of aspartic proteases.

4. The chimeric polynucleotide of claim 1, wherein said proteolytic enzyme is a chymosin.

5. The chimeric polynucleotide of claim 1, wherein any one of said caseins is a bovine casein.

6. The chimeric polynucleotide of claim 1, wherein said whey polypeptide is a bovine or a fungal whey polypeptide.

7. The chimeric polynucleotide of claim 1, comprising a codon optimized sequence for expression in a cell.

8. The chimeric polynucleotide of claim 7, wherein said cell is a transfected or a transgenic cell.

9. The chimeric polynucleotide of claim 1, comprising the nucleotide sequence set forth in any one of SEQ ID Nos: 37-42.

10. A plasmid comprising the chimeric polynucleotide of claim 1.

11. The plasmid of claim 10, wherein said plasmid is an expression vector.

12. A chimeric polypeptide encoded, expressed, or both from: the chimeric polynucleotide of claim 1.

13. The chimeric polypeptide of claim 12, comprising the amino acid sequence set forth in any one of SEQ ID Nos: 16-21.

14. A cell, comprising the chimeric polynucleotide of claim 1.

15. A composition comprising the chimeric polypeptide of claim 12, and an acceptable carrier.

16. The composition of claim 15, further comprising any one of:
calcium salt, magnesium salt, and both.

17. The composition of claim 15, being an edible composition, and optionally wherein said edible composition is a dairy product substituent.

18. (canceled)

19. A method for producing an edible composition comprising casein and/or whey polypeptide aggregates, the method comprising providing a composition comprising a chimeric polypeptide comprising: (i) a first polypeptide being a polypeptide selected from the group consisting of: alpha S1 casein, alpha S2 casein, beta casein, whey polypeptide, whey polypeptide, and a functional analog thereof; and (ii) a second polypeptide being a kappa casein macropeptide, wherein said first polypeptide and said second polypeptide are interspaced by a cleavage site of a proteolytic enzyme, and contacting said composition with an effective amount of said proteolytic enzyme, thereby producing the edible composition comprising casein and/or whey polypeptide aggregates.

20. The method of claim 19, wherein: (i) said method further comprises a step proceeding said contacting, comprising isolating said casein and/or whey polypeptide aggregates; (ii) said contacting is performed at pH of 4.0-6.0; (iii) said produced edible composition comprising said aggregates of casein and/or whey polypeptide, comprises a cheese or a curd; or (iv) any combination of (i) to (iii).

21.-22. (canceled)

23. An edible composition produced according to the method of claim 19.
